(19) United States
(12) Patent Application Publication
(10) Pub. No.: US 2012/0245201 A1
(43) Pub. Date: Sep. 27, 2012

(54) Isopropylphenidate for Treatment of Attention-Deficit/Hyperactivity Disorder and Fatigue-Related Disorders and Conditions

(76) Inventors: John S. Markowitz, Gainesville, FL (US); Kennerly S. Patrick, Mt. Pleasant, SC (US); Haojie Zhu, Gainesville, FL (US)

(21) Appl. No.: 13/384,920

(22) PCT Filed: Jul. 21, 2010
(86) PCT No.: PCT/US10/42769

§ 371 Date: Jun. 14, 2012

(30) Foreign Application Priority Data
Jul. 23, 2009 (US) 61227876

(51) Int. Cl. A61K 31/4458; A61P 25/20; A61P 25/18; A61P 25/00; A61P 25/04

(52) U.S. Cl. 514/317

(57) ABSTRACT

The present invention relates to the identification of isopropylphenidate as a useful therapeutic agent in the treatment of Attention-Deficit/Hyperactivity Disorder (ADHD) as well as various fatigue causing disease and disorders and medication induced fatigue.

The present application claims benefit of priority to U.S. Provisional Application Ser. No. 61/227,876 filed Jul. 23, 2009, the entire contents of which are hereby incorporated by reference.

### BACKGROUND OF THE INVENTION

1. Field of the Invention

The present invention relates generally to the fields of neurobiology and pharmacology. More particularly, it concerns the use of d- or dl-threo-isopropylphenidate (d- or dl-threo-α-phenyl-2-piperidineacetic acid isopropyl ester) or salts thereof in the treatment of attention-deficit/hyperactive disorder (ADHD) and a variety of neurological and fatigue disorders.

2. Description of Related Art

Attention-deficit/hyperactivity disorder (ADHD) is a complex neurobehavioral disorder characterized by varying degrees of inattention, hyperactivity, and impulsivity (Biederman, 2005). It is perhaps the single most common chronic health problems afflicting school-age children with an estimated worldwide prevalence of 8-12% (Faraone, 2003). The most widely utilized pharmacological treatment of ADHD is the psychostimulant methylphenidate (MPH) consisting of the racemic (50:50) mixture of d-threo-(R,R)-MPH and 1-threo-(S,S)-MPH isomers (FIG. 1). Depicted in bold within these structures is the phenethylamine pharmacophore of d-MPH shared by the neuronal substrates dopamine (DA) and norepinephrine (NE) (Patrick and Markowitz, 1997). Methylphenidate is an effective and well-tolerated treatment that has been in general clinical use for over 50 years. dl-MPH is widely available as an immediate-release tablet and displays a short plasma half-life of 2-3 hrs in this formulation as a result of rapid and stereoselective metabolism via deesterification (see Patrick et al., 2005). This hydrolytic process yields the major, albeit inactive, metabolite ritalinic acid (RA) that typically attains blood concentrations 30-60 times those of the parent compound (Patrick and Markowitz, 1997).

Use of immediate-release formulations of MPH result in a relatively brief duration of action necessitating multiple daily doses to achieve symptom control throughout the day (Markowitz et al., 2003; Patrick et at 2005). However, administering MPH throughout the day poses multiple problems related to convenience, security, and finally, patient self-esteem as a consequence of the need for medication dosing in the presence of peers and others, particularly for the school-age child (Greenhill et al., 2002). Accordingly, the most common clinical practice in the US is to prescribe one of several extended-release (ER) or so-called modified-release pharmaceutical formulations (Markowitz et al., 2003) that vary from one another only in the duration of action which is dependent upon total dose per tablet/capsule/transdermal patch, and the MPH release rate and pattern (i.e., proportion of total dosage form released over the dosing interval which is generally once every 24 hrs). Although the fundamental half-life (t_{1/2}) of the MPH molecule remains unchanged upon release from these formulations, this approach can provide the necessary all-day “coverage” of symptoms. Despite these advances in biopharmaceutical delivery of MPH, in many instances these formulations are not successful for patients due to inter-individual physiological differences in drug absorption as such ER formulations pass the various regions of the gut (McConnell et al., 2008), well recognized inter-individual differences in metabolism resulting in non-response or intolerable adverse effects that may be genetically based (Zhu et al., 2008), or suboptimal pharmacodynamic responses even when adequate doses and/or systemic concentrations are known to be achieved through blood sample monitoring studies. Further limitations of the existing formulations include the high out-of-pocket expense of ER dosage forms, or lack of coverage or limited coverage extended through third party prescription insurance plans or state/federal prescription coverage plans/formularies.

A large and diverse group of analogues of MPH (e.g., dl-threo-α-(2-piperidyl)-phenylacetic acid ester homologs) have been synthesized and there has been some, albeit limited pharmacological investigation of a number of these agents. The primary central nervous system (CNS) target of interest has been the DA transporter (DAT) (Singh, 2000). Structural modifications have been made to the known and existing MPH molecule have been undertaken by other researchers and these can generally be divided into phenyl ring substitutions, alterations to the piperidine ring, and a homologous series of alkyl ester congeners (Thai et al., 1999; Singh, 2000).

It would be a significant clinical advance in the field of ADHD study and treatment if an MPH-like medication could be identified and clinically developed which exerted both therapeutic effects equal or superior to MPH within the (CNS), while simultaneously exhibiting more desirable attributes including but not limited to a longer half-life relative to MPH due to greater resistance to the primary metabolic pathway, de-esterification by the major hepatic enzyme in humans, carboxylesterase-1 (hCES1), the ultimate production of non-toxic metabolites (Patrick et al., 1981), and a superior side effect profile improving tolerability (e.g., reduced cardiovascular side effects such as elevation of blood pressure and heart rate) and hence compliance. Furthermore, a potentially a decreased liability to interact with other drugs or chemicals as well as a less drastic pharmacokinetic alteration and pharmacodynamic response in the presence of CES1 genetic polymorphisms (Zhu et al., 2008) would be highly advantageous. These attributes could potentially circumvent the need for the complex ER stimulant dosage forms formulated as long-acting slow-release products in wide use today (Markowitz et al., 2003). Further, these other potential attributes described for an alternative molecule or derivative could elevate the overall safety margin over that of the presently available therapeutic agents for ADHD.

### SUMMARY OF THE INVENTION

Thus, in accordance with the present invention, there is provided a method of treating a subject with attention-deficit/hyperactivity disorder (ADHD) comprising administering to the subject a pharmaceutically acceptable form of d- and/or dl-threo-isopropylphenidate or a salt thereof. The subject is a human, such as a child or adolescent, or an adult. The subject may further suffer from a comorbid disorder. The subject may be treated with a second ADHD therapy, such as methylphenidate.

The d- and/or dl-threo-isopropylphenidate or salt thereof may be administered to the subject in a unit dosage form of 1 to 200 milligrams, or more particularly, about 10 to 100 milligrams, and about 25-50 mg. The subject may be provided the unit dosage form every 4-24 hours. The d- and/or dl-threo-isopropylphenidate or salt thereof is administered orally or parenterally. The d- and/or dl-threo-isopropylphenidate or salt thereof may be administered as an injectable solution, a transdermal patch, a capsule, a tablet, an intranasal or sublingual spray, a syrup, or a solution.

The subject may consume alcohol during treatment with d- and/or dl-threo-isopropylphenidate or salt thereof. The subject may be comprised of cells having a mutated human carboxylesterase-1 gene. The d- and/or dl-threo-isopropylphenidate or salt thereof may be substantially d-isopropylphenidate or substantially dl-isopropylphenidate, or may be a racemic mixture. The d- and/or dl-threo-isopropylphenidate salt may be a hydrochloride, hydrobromide, sulfate, aspartate, saccharate, succinate, tartrate, mesylate, or palmitate salt.

In another embodiment, there is provided a pharmaceutical formulation comprising (a) d- and/or dl-threo-isopropylphenidate or salt thereof and (b) an opiate and/or a sedating anti-psychotic. The formulation may be an injectable solution, a transdermal patch, a capsule, a tablet, an intranasal or sublingual spray, a solution or a syrup. Still another embodiment provides a pharmaceutical formulation comprising (a) d- and/or dl-threo-isopropylphenidate or salt thereof and (b) methylphenidate. The formulation may be an injectable solution, a transdermal patch, a capsule, a tablet, an intranasal or sublingual spray, a solution or a syrup.

In yet another embodiment, there is provided a method of treating fatigue or somnolence in a subject comprising administering to the subject a pharmaceutically acceptable form of d- and/or dl-threo-isopropylphenidate or salt thereof. The subject may be a human, such as an adult. The fatigue or somnolence may be drug-induced fatigue, such as by a sedating anti-psychotic or an opiate. The fatigue or somnolence may be caused by a sleep disorder, such as narcolepsy, obstructive sleep apnea/hypopnea syndrome, or shiftwork sleep disorder. The fatigue or somnolence may be caused by a disease, such as depression, apathy, multiple sclerosis or Parkinson's Disease.

The d- and/or dl-threo-isopropylphenidate or salt thereof may be administered to the subject in a unit dosage form of 1 to 200 milligrams, or more particularly, about 10-100 milligrams, and about 25-50 mg. The subject may be provided the unit dosage form every 4-24 hours. The d- and/or dl-threo-isopropylphenidate or salt thereof may be administered orally. The d- and/or dl-threo-isopropylphenidate or salt thereof may be administered as an injectable solution, a transdermal patch, capsule, a tablet, an intranasal or sublingual spray, a solution or a syrup.

The method subject may consume alcohol during treatment with isopropylphenidate. The subject may be comprised of cells having a mutated human carboxylesterase-1 gene. The d- and/or dl-threo-isopropylphenidate or salt thereof may be substantially d-isopropylphenidate or may be substantially dl-isopropylphenidate, or may be a racemic mixture. The d- and/or dl-threo-isopropylphenidate salt may be a hydrochloride, hydrobromide, sulfate, aspartate, saccharate, succinate, tartrate, mesylate, or palmitate salt.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Structures for methylphenidate (MPH) and isopropylphenidate (IPH).

FIG. 2—Hydrolysis of methylphenidate (MPH) and isopropylphenidate (IPH) by recombinant human CES1, HLM, HIM. After incubation with CES 1 transfected human cells, HLM, and HIM at 37° C. for 60 min, the metabolite RA produced via hydrolysis of MPH and IPH was determined by HPLC. Data are presented as mean±SEM of 3 to 6 independent experiments.

FIG. 3—Transesterification potential of isopropylphenidate (IPH) versus methylphenidate (MPH) by s9 fractions of CES1 transfected cells in the presence of ethanol. MPH (1 mM) or IPH (1 mM) was incubated with s9 fractions of hCES1 overexpressing cells (2 mg/ml) in the presence or absence of ethanol (10 mM) at 37° C. for 30 min. The transesterification product EPH and hydrolytic metabolite RA were analyzed utilizing established HPLC assays. IPH is shown to be a poor substrate of both hydrolysis and transesterification reactions catalyzed by hCES1. Furthermore, no EPH formation was observed following the incubation of MPH and IPH with ethanol when hCES1 was not present (data not shown). Data are expressed as mean±SEM of three independent experiments.

FIGS. 4A-B—Locomotor activity of SD rats after i.p. injection of IPH (10 mg/kg) and saline. The time course of locomotor response over a 120 min period following i.p. administration of IPH (10 mg/kg) versus saline is shown in FIG. 4A. As expected, IPH produced robust effects on locomotor activity in rats compared to saline injections at every 10 min period recorded post-dosing with a mean of nearly 1200 counts recorded during the initial 10 min measurement. Shown in FIG. 4B are the cumulative locomotor activity counts over the entire 120 min study period. In summary, racemic IPH administration significantly elevated the cumulative locomotor activity counts in comparison to saline-injected rats (p<0.01).

### DESCRIPTION OF ILLUSTRATIVE EMBODIMENTS

As discussed above, ADHD is a complex neurobehavioral disorder characterized by varying degrees of inattention, hyperactivity, and impulsivity and is perhaps the single most common chronic health problems afflicting school-age children with an estimated worldwide prevalence of 8-12%. The most widely utilized pharmacological treatment of ADHD is the psychostimulant methylphenidate (MPH), which is an effective and well-tolerated treatment that has been in general clinical use for over 50 years. Unfortunately, immediate-release formulations of MPH result in a relatively brief duration of action necessitating multiple daily doses to achieve symptom control throughout the day, while administering MPH throughout the day poses problems related to convenience, security, and patient self-esteem, particularly for the school-age child. And despite advances in biopharmaceutical delivery of MPH, in many instances these formulations are not successful for patients due to inter-individual physiological differences in drug absorption as such ER formulations pass the various regions of the gut, are too expensive, or their use is not covered by third party prescription plans including some private insurance plans as well as some state Medicaid formularies. Many analogues of MPH (dl-α-(2-piperidyl)-phenylacetic acid ester homologs) have been synthesized and their pharmacology investigated in the hopes of finding a longer acting analog of sufficient structural similarity to MPH would be metabolized almost entirely to the same major inactive, non-toxic metabolite (i.e., R A; Patrick et al., 1981), as MPH.

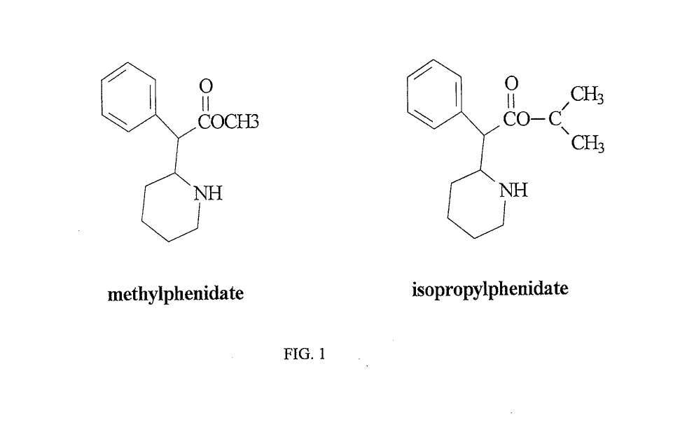

The present study tested the isopropyl ester derivative of MPH, isopropylphenidate (IPH) (FIG. 1) for its ability to exert prolonged pharmacologic activity relative to MPH due to the presence of the bulkier isopropyl group which imparts resistance to enzyme (hCES1) mediated metabolic catalysis. Much earlier studies, i.e., those performed over 45 years ago, suggested that IPH was much more resistant to either acid- or base-catalyzed (i.e., chemical) hydrolysis relative to MPH (Portoghese and Malspeis, 1961). The inventors now show that this enhanced resistance to chemical hydrolysis also generalizes to resistance toward enzymatic hydrolysis via hepatic hCES1. In view of hCES1-mediated ester hydrolysis being the widely recognized and primary biotransformation pathway terminating the action of MPH, a slower in vivo hydrolysis of IPH relative to MPH would lead to an extended duration of action providing a resultant therapeutic benefit over existing MPH formulations in the treatment of ADHD. Regarding the neuropharmacology and hypothesized therapeutic effects of IPH, an early in vitro assay measuring the inhibition of [³H]-(±)-MPH binding to rat brain striatal synaptosomes found the binding of the racemic isopropyl derivative (i.e. IPH) to exhibit approximately one third the activity of the methyl ester (i.e., MPH) based upon a mg/mg dosage comparison (Schweri et al., 1985). However, the earliest assessment of IPH pharmacological activity was in vivo, where its recorded activity in antagonizing the physiological influences of reserpine pre-treatment in dogs were compared against that of MPH and found to be of lower potency (Portoghese and Malspeis, 1961).

However, it is of paramount importance to recognize the confounding influence of the reserpine pre-exposure employed in this previous experiment's design as a pharmacological probe to destroy pre-synaptic catecholamine vesicles. That is, the presence of intact catecholaminergic vesicles are now accepted as both necessary and fundamental to the pharmacological mechanism of action by which such drugs as MPH produce their psychostimulant/psychomotor effects. Thus, reserpinization most certainly confounds if not obviates any extrapolation relative to the dopaminergic and noradrenergic effects of IPH versus MPH at the pre-synaptic transporter, i.e., the well-established site of ADHD therapeutic action (Markowitz and Patrick, 2008).

Not withstanding these previous reports, their findings, and the aforementioned limitations of these earlier study designs, the isopropyl ester derivative of MPH, IPH was newly synthesized and subsequently rigorously assessed utilizing modern pharmacological “lead compound” screening methodologies directed at laying the foundation of IPH as a potential therapeutic agent for ADHD and other neuropsychiatric disorders. State-of-the-art monoamine transporter binding and cellular uptake experiments were performed assessing racemic isopropylphenidate (dl-IPH) as well as the present gold standard comparator dl-MPH. Additionally, an in vitro assessment of the extent and rate of metabolic hydrolysis of both compounds via hCES1.was performed utilizing unique human hCES 1 transfected cell lines developed in the inventors' laboratory.

Finally, an in vivo assessment of IPH stimulant effects (versus a placebo treatment, saline) was performed measuring rodent locomotor activity, a standard behavioral indicator of psychostimulant activity. This experiment utilized the identical experimental methodology previously employed in a widely cited study establishing in which the in vivo locomotor activity and pharmacology of the separate d- and l-isomers of MPH in the rat (Patrick et al., 1987). The overarching goal of this step-wise series of experiments was to further define the pharmacological characteristics of IPH that establish its potential as a therapeutic agent.

The inventors have now demonstrated that the binding of IPH and MPH to the prominent cellular monoamine transporters DAT, NET, and SERT revealed that the affinity was greatest towards DAT, and that both tested compounds produced significant effects at the DAT with insignificant differences noted between the two compounds. Furthermore, with regard to NET, it was noted that IPH exhibited substantially less binding affinity than MPH which was significantly higher. Uptake of NE was noted to be significantly lower for IPH relative to MPH.. Lastly, 5-HT uptake was not significantly influenced by either of the agents.

The results of complementary cellular functional studies indicated that both tested agents produced a high degree of DA uptake inhibition with little difference in this action noted between the two compounds tested. Neither of the tested compounds was found to exert any significant activity at SERT.

With regard to hydrolysis of these respective esters, experimental results indicated that the catalytic efficiency of hCES1-mediated hydrolysis towards MPH was approximately 10-fold higher than for IPH. Thus, IPH is a poor substrate of hCES1, but though it is ultimately metabolized, albeit at a markedly lower rate, by hCES1 with essentially no contribution from other hydrolytic enzymes such as hCES2.

Importantly, it should be noted that IPH is ultimately metabolized to the same major albeit pharmacologically inactive (Patrick et al., 1981) metabolite as MPH, i.e., ritalinic acid, and hence IPH will not be subject to yielding any uncharacterized metabolites that might otherwise be a source of concern due to a potential to produce adverse effects and/or toxicities. IPH was also noted to be a poor substrate of transesterification reactions known to be catalyzed by hCES1.

In locomotor testing, as with MPH, IPH was also observed to produce robust effects on locomotor activity in rats relative to control animals administered saline injections with data collections/counts recorded at 10 min time intervals post-dosing with a mean of nearly 1200 counts recorded during the initial 10 min measurement.

Thus, in all major domains assessed, IPH constitutes a promising alternative to the current and widespread use of MPH as a foundational pharmacological treatment—alone or as a combination with other drugs or therapies—for ADHD. Moreover, with the growing recognition of the continuation of ADHD into adulthood, increased risk in this demographic for alcohol use or use disorder, as well as stimulant abuse potential and continued concern over cardiovascular events associated with MPH and other psychostimulants in the biomedical literature (see Patrick et al., 2009), makes the development of alternative treatments an even greater imperative. Indeed, the lower noradrenergic activity associated with IPH relative to that observed with MPH in the pharmacologic screening studies conducted by the inventors suggests it may be a more desirable compound given the more common association of perturbations of NE rather than DA with adverse cardiovascular events. The established hCES1-based drug interaction (i.e., transesterification) of MPH and alcohol discovered by the inventors' group (Markowitz et al., 1999, 2000) appears to be avoidable with the IPH compound that is a much poorer hCES1 substrate relative to MPH. Observed results suggest that its clinical use would be likely to reduce the risk of such drug-drug interactions substantially. These and other aspects of the invention are described in detail below.

### I. ADHD

A. Background

Attention-deficit/hyperactivity disorder (AD/HD or ADHD) is a neurobehavioral developmental disorder. It is the most commonly diagnosed psychiatric disorder in children affecting about 3 to 7% of children globally with symptoms starting before seven years of age. It is characterized by a persistent pattern of impulsiveness and inattention, with or without a component of hyperactivity. ADHD is diagnosed twice as frequently in boys as in girls, though studies suggest this discrepancy may be due to subjective bias. ADHD is generally a chronic disorder with 30 to 50% of those individuals diagnosed in childhood continuing to have symptoms into adulthood. As they mature, adolescents and adults with ADHD are likely to develop coping mechanisms to compensate for their impairment.

Though previously regarded as a disorder limited to childhood and adolescence, it is now widely accepted that ADHD can continue throughout adulthood. Four percent of American adults are estimated to live with ADHD. ADHD management usually involves some combination of medications, behavior modifications, life-style changes, or counseling. Untreated adults with ADHD often have chaotic life-styles, may appear to be disorganized and may rely on non-prescribed drugs and alcohol to get by. They often have such associated psychiatric comorbidities as depression, anxiety, bipolar disorder, alcohol use disorder, substance use disorder, or a learning disability. A diagnosis of ADHD may offer adults insight into their behaviors and allow patients to become more aware and seek help with coping and treatment strategies.

Only recognized as occurring in adults in 1978, it is currently not addressed separately. Obstacles that clinicians face when assessing adults who may have ADHD include developmentally inappropriate diagnostic criteria, age-related changes, comorbidities and the possibility that high intelligence or situational factors can mask ADHD symptoms. The most common symptoms of ADHD are impulsiveness; hyperactivity; and inattention. The Diagnostic and Statistical Manual of Mental Disorders, 4^{th} revision (DSM-IV) categorizes the symptoms of ADHD into three clusters, referred to as subtypes: (1) inattentive; (2) hyperactive/impulsive; and (3) combined. Most people exhibit some of these behaviors but not to the point where they significantly interfere with a person's work, relationships, or studies.

ADHD may accompany other disorders such as anxiety or depression, which can greatly complicate diagnosis and treatment. Published studies suggest that depression in conjunction with ADHD appears to be increasingly prevalent in children as they get older, with a higher rate of increase noted in girls than boys, and to vary in prevalence with the subtype of ADHD. When a mood disorder complicates the presentation of ADHD it is believed to be be prudent to treat the mood disorder first; however, parents of children diagnosed with ADHD often wish to have the ADHD treated first, because the response to treatment is much quicker relative to the pharmacotherapies available for depression. Hyperactivity is common among children with ADHD but tends to disappear during adulthood. However, over half of children with ADHD continue to have some symptoms of inattention throughout their lives.

B. Diagnosis

ADHD and its diagnosis and treatment have been considered controversial since the 1970s. The controversies have involved clinicians, teachers, policymakers, parents and the media, with opinions regarding ADHD that range from not believing it exists at all to believing there are genetic and physiological bases for the condition and also include disagreement about the use of stimulant medications in treatment. However, the major psychostimulants in clinical use today including MPH and amphetamine preparations produce one of the most robust and rapid clinical responses relative to essentially every therapeutic class of agents (e.g., antidepressants, anxiolytics) in all of psychopharmacology. Most healthcare providers accept that ADHD is a genuine disorder with debate in the scientific community mainly around how it is diagnosed and treated.

ADHD may be seen as an extreme of one or more continuous traits found throughout the population. ADHD is a developmental disorder in which certain traits such as impulse control lag in development when compared to the general population. Using magnetic resonance imaging of the prefrontal cortex, this developmental lag has been estimated to range from 3 to 5 years. These delays are considered to cause impairment. ADHD has also been classified as a behavior disorder.

ADHD is classified as a disruptive behavior disorder along with oppositional defiant disorder, conduct disorder and antisocial disorder. Inattention and “hyperactive” behavior are not the only problems in children with ADHD. ADHD exists alone in only about ⅓ of the children diagnosed with it. The co-existence of other neuropsychiatric disorders is extremely common in individuals with ADHD and require individual assessment, diagnosis, and treatment rather than being viewed as part of the general ADHD diagnosis and associated symptoms. Some of the more common associated conditions include but are not limited to: oppositional defiant disorder and conduct disorder, which both are characterized by anti-social behaviors such as stubbornness, aggression, frequent temper tantrums, deceitfulness, lying, or stealing; primary disorder of vigilance; mood disorders; biopolar disorder; and a variety of anxiety disorders. Each of these can be amenable to pharmacological treatment as well when behavioral interventions or psychotherapy are inadequate. Consequently, clinicians must be cognizant of the potential for drug-drug interactions if medications are added to an existing medication regimen prescribed for the treatment of ADHD.

C. Risk Factors

Twin studies indicate that the disorder is highly heritable and that genetics are a factor in about 75% of ADHD cases. Hyperactivity also seems to be primarily a genetic condition; however, other causes do have an effect. Researchers believe that a large majority of ADHD cases arise from a combination of various genes, many of which affect dopamine transporters. Candidate genes include dopamine transporter, dopamine receptor D4, dopamine β-hydroxylase, monoamine oxidase B, catecholamine-methyl transferase, serotonin transporter promoter (SLC6A4), 5-hydroxytryptamine 2A receptor (5-HT2A), 5-hydroxytryptamine 1B receptor (5-HT1B), the 10-repeat allele of the DAT1 gene, the 7-repeat allele of the DRD4 gene, and the dopamine β hydroxylase gene (DBH TaqI). The broad selection of targets suggests that ADHD does not follow the traditional model of a “genetic disease” and should therefore be viewed as a complex interaction among genetic and environmental factors. Even though all of these genes and others may constitute individual and perhaps combined risk factors, to date no single gene or gene variant has been demonstrated to substantially contribute to the development of ADHD. Nevertheless, active genetic research is ongoing in this area. Twin studies to date have also suggested that approximately 9% to 20% of the variance in hyperactive-impulsive-inattentive behavior or ADHD symptoms can be attributed to nonshared environmental (nongenetic) factors.

Environmental factors implicated include alcohol and tobacco smoke exposure during pregnancy and environmental exposure to lead in very early life. The relation of smoking to ADHD could be due to nicotine causing hypoxia (lack of oxygen) to the fetus in utero. It could also be that women with ADHD are more likely to smoke and therefore, due to the strong genetic component of ADHD, are more likely to have children with ADHD. Complications during pregnancy and birth, including premature birth, might also play a role.

The European Food Safety Authority (EFSA) reviewed the literature on the association between food additives and hyperactivity and concluded that there is only limited evidence of an association between the intake of additives and activity and attention and then only in some children studied. They further indicated that the effects reported in the study were not consistent for the two age groups and for the two food additive mixtures used in the study. Others have suggested a trial of removing additives from the diet for children with ADHD as it is harmless and might be helpful. A number of studies have found that sucrose (sugar) has no effect on behavior and in particular it does not exacerbate the symptoms of children diagnosed with ADHD. Thus neither food additives or sugar consumption have been shown to significantly influence behavioral symptoms in any adequately powered placebo-controlled, double blinded study of ADHD.

Furthermore, there is no compelling evidence that social factors alone can cause ADHD. However, many researchers believe that relationships with caregivers have a profound effect on attentional and self-regulatory abilities. A study of foster children found that a high number of them had symptoms closely resembling ADHD, while other researchers have found behavior typical of ADHD in children who have suffered violence and emotional abuse. Furthermore, Complex Post Traumatic Stress Disorder can result in attention problems that can look like ADHD. ADHD is also considered a contributing factor to Sensory Integration Disorders.

D. Pathophysiology

The pathophysiology of ADHD is unclear and there are a number of competing theories. Neuroimaging studies in ADHD have not always given consistent results and as of 2008 are only used for research purposes. In one study, a delay in development of certain brain structures by an average of three years occurred in ADHD elementary school aged patients. The delay was most prominent in the frontal cortex and temporal lobe, which are believed to be responsible for the ability to control and focus thinking In contrast, the motor cortex in the ADHD patients was seen to mature faster than normal, suggesting that both slower development of behavioral control and advanced motor development might be required for the fidgetiness that characterizes ADHD. It should be noted that stimulant medication itself can affect growth factors of the central nervous system.

The same laboratory had previously found involvement of the “7-repeat” variant of the dopamine D4 receptor gene, which accounts for about 30 percent of the genetic risk for ADHD, in unusual thinness of the cortex of the right side of the brain; however, in contrast to other variants of the gene found in ADHD patients, the region normalized in thickness during the teen years in these children, coinciding with clinical improvement.

Additionally, SPECT scan results have suggested that individuals with ADHD appear to have reduced blood circulation (indicating low neural activity), and a significantly higher concentration of DAT in the striatum, an area which may be viewed in general terms as in charge of planning ahead. A study by the U.S. Department of Energy's Brookhaven National Laboratory in collaboration with Mount Sinai School of Medicine in New York suggest that it is not the DAT density that are biomarkers of ADHD, but the brain's ability to produce DA itself. The study was done by injecting 20 ADHD subjects and 25 control subjects with a radiotracer that attaches itself to DAT. ADHD subjects were found to exhibit lower concentrations of DA overall. The investigators speculated that since ADHD subjects had lower levels of DA to begin with, the number of transporters in the brain was not the revealing factor. In support of this notion, plasma homovanillic acid, an index of DA concentrations or turnover, was found to be inversely related not only to childhood ADHD symptoms in adult psychiatric patients, but to “childhood learning problems” in healthy subjects as well.

Although there is evidence for DA abnormalities in ADHD, it is not clear whether abnormalities of the dopaminergic system constitute the neurobiological basis for the ADHD phenotype or merely represent a finding which is a consequence of CNS dysfunction elsewhere. Researchers have also described a form of ADHD in which the abnormality appears to be sensory overstimulation resulting from a disorder of ion channels in the peripheral nervous system.

E. Diagnosis

No objective test exists to make a diagnosis of ADHD. It thus remains a clinical diagnosis. In North America, the Diagnostic and Statistical Manual of Mental Disorders, 4^{th} Ed. (DSM-IV) criteria are often the basis for a diagnosis, while European countries usually favor the ICD-10. Many of the symptoms of ADHD occur from time to time in everyone; in patients with ADHD, the frequency of these symptoms is greater and significantly impairs their life. This impairment must occur in multiple settings to be classified as ADHD. As with many other psychiatric and medical disorders, the formal diagnosis is made by a qualified professional in the field based on a set number of criteria.

In the U.S., these criteria are laid down by the American Psychiatric Association in their DSM-IV. Based on the DSM-IV criteria listed below, three types of ADHD are classified:
ADHD, Combined Type: if both criteria 1A and 1B are met for the past 6 months ADHD Predominantly Inattentive Type: if criterion 1A is met but criterion 1B is not met for the past six months
ADHD, Predominantly Hyperactive-Impulsive Type: if Criterion 1B is met but Criterion 1A is not met for the past six months.

The previously used term ADD expired with the most recent revision of the DSM. Consequently, ADHD is the current nomenclature used to describe the disorder as one distinct disorder which can manifest itself as being a primary deficit resulting in hyperactivity/impulsivity (ADHD, predominately hyperactive-impulsive type) or inattention (ADHD predominately inattentive type) or both (ADHD combined type).

In the tenth edition of the International Statistical Classification of Diseases and Related Health Problems (ICD-10) the symptoms of ADHD are given the name “Hyperkinetic disorders.” When a conduct disorder (as defined by ICD-10) is present, the condition is referred to as “Hyperkinetic conduct disorder.” Otherwise the disorder is classified as “Disturbance of Activity and Attention,” “Other Hyperkinetic Disorders” or “Hyperkinetic Disorders, Unspecified.” The latter is sometimes referred to as, “Hyperkinetic Syndrome.”

The American Academy of Pediatrics Clinical Practice Guideline for children with ADHD emphasizes that a reliable diagnosis is dependent upon the fulfillment of three criteria:
the use of explicit criteria for the diagnosis using the DSM-IV-TR;
the importance of obtaining information about the child's symptoms in more than one setting;
the search for coexisting conditions that may make the diagnosis more difficult or complicate treatment planning
   All three criteria are determined using the patient's history given by the parents, teachers and/or the patient. Adults often continue to be impaired by ADHD. Adults with ADHD are diagnosed under the same criteria, including the stipulation that their symptoms must have been present prior to the age of seven. Adults face some of their greatest challenges in the areas of self-control and self-motivation, as well as executive functioning, usually having more symptoms of inattention and fewer of hyperactivity or impulsiveness than children do.

Common comorbid conditions include oppositional defiant disorder (ODD). About 20% to 25% of children with ADD meet criteria for a learning disorder. Learning disorders are more common when there are inattention symptoms. Comorbid disorders or substance abuse can make the diagnosis and treatment of ADHD more difficult. Psychosocial therapy is useful in treating some comorbid conditions. Depression may also coincide with ADHD, increasingly prevalent among girls and older children. Epilepsy is a commonly found comorbid disorder in ADHD diagnosed individuals. Some forms of epilepsy can also cause ADHD like behaviour which can be misdiagnosed as ADHD.

To make the diagnosis of ADHD, a number of other possible medical and psychological conditions must be excluded. Medical conditions that must be excluded include hypothyroidism, anemia, lead poisoning, chronic illness, hearing or vision impairment, substance abuse, medication side effects, sleep impairment and child abuse, among others.

Among other psychological and neurological issues, the relationship between ADHD and sleep is complex. In addition to clinical observations, there is substantial empirical evidence from a neuroanatomic standpoint to suggest that there is considerable overlap in the central nervous system centers that regulate sleep and those that regulate attention/arousal. Primary sleep disorders play a role in the clinical presentation of symptoms of inattention and behavioral dysregulation. There are multilevel and bidirectional relationships among sleep, neurobehavioral functioning and the clinical syndrome of ADHD.

Behavioral manifestations of sleepiness in children range from classic manifestations (yawning, rubbing eyes), to externalizing behaviors (impulsivity, hyperactivity, aggressiveness), to mood lability and inattentiveness.

From a clinical standpoint, mechanisms that account for the phenomenon of excessive daytime sleepiness include chronic sleep deprivation, that is insufficient sleep for physiologic sleep needs, fragmented or disrupted sleep, caused by, for example, obstructive sleep apnea (OSA) or periodic limb movement disorder (PLMD), primary clinical disorders of excessive daytime sleepiness, such as narcolepsy and circadian rhythm disorders, such as delayed sleep phase syndrome (DSPS). All of these are important causes of symptoms which may overlap with the cardinal symptoms of ADHD and children with ADHD should be regularly and systematically assessed for sleep problems.

F. Management and Prognosis

Methods of treatment often involve some combination of behavior modification, medication, life-style changes and counseling. Combined medical management and behavioral treatment is the most effective ADHD management strategy, followed by medication alone, and then behavioral treatment.

In terms of cost-effectiveness, management with medication has been shown to be the most cost-effective, followed by behavioral treatment and combined treatment. Stimulants are the most commonly prescribed medications for ADHD. The most common stimulant medications are methylphenidate (Ritalin®, Metadate®, Concerta®), dextroamphetamine (Dexedrine®), d-methamphetamine (Desoxyn®) and mixed amphetamine salts (Adderall®). Atomoxetine (Strattera®) is currently the only non-stimulant drug approved for the treatment of ADHD. Other medications that may be prescribed off-label include certain antidepressants such as tricyclic antidepressants, serotonin-norepinephrine reuptake inhibitors (SNRIs) or MAOIs. One 2007 drug class review suggested there are few adequate studies of comparative effectiveness between various drugs for ADHD and that there is a lack of quality evidence on their effects on overall academic performance and social behaviors. The long term effects of ADHD medications in preschool children are unknown and are not recommended for pre-school children.

Stimulants used to treat ADHD raise the extracellular concentrations of the neurotransmitters DA and NE which causes an increase in neurotransmission. The therapeutic benefits are due to noradrenergic effects at the locus coeruleus and the prefrontal cortex and dopaminergic effects at the nucleus accumbens.

Although “under medical supervision, stimulant medications are considered safe,” the use of stimulant medications for the treatment of ADHD has generated controversy because of undesirable side effects, uncertain long term effects and social and ethical issues regarding their use and dispensation. The FDA has added black-box warnings to some ADHD medications. The American Heart Association and the American Academy of Pediatrics feels that it is prudent to carefully assess children for heart conditions before treating them with stimulant medications. The potential for adverse drug reactions as a result of drug-drug interactions (DDIs) also remains an area of ongoing concern for all therapeutic categories of medications. The importance of adverse drug reactions (ADRs) which encompass DDIs has been extensively documented. Medications inadvertently result in significant patient morbidity, mortality, and significantly contribute to ever increasing costs of medical care, morbidity, and mortality. An estimated 2 million hospitalized patients have severe ADRs annually in the U.S., even when drugs are appropriately prescribed and administered. ADRs purportedly ranked between the 4^{th} and 6th leading causes of death in the U.S. in 1994 (Lazarou et al., 1998). In a recently published study, ADRs purportedly affected nearly 2.2 million individuals and caused over 100,000 deaths making them one of the leading causes of hospitalization and death in the US. The economic implications of medication “misadventures” are enormous with estimates approaching $180 billion annually. Further, ADRs including DDIs or the potential thereof are the leading cause of market withdrawal of FDA-approved drugs.

Psychological therapies use to treat ADHD include psychoeducational input, behavior therapy, cognitive behavioral therapy (CBT), interpersonal psychotherapy (IPT), family therapy, school-based interventions, social skills training and parent management training

Parent training and education have been found to have short term benefits. Family therapy has shown to be of little use in the treatment of ADHD. Though it may be worth noting that parents of children with ADHD are more likely to divorce than parents of children without ADHD, particularly when their children are under 8 years of age. Several ADHD specific support groups exist as informational sources and to help families cope with challenges associated with dealing with ADHD.

Dietary supplements and specialized diets are sometimes used by people with ADHD with the intent to mitigate some or all of the symptoms. For example, omega-3 supplementation may reduce ADHD symptoms for a subgroup of children and adolescents with ADHD “characterized by inattention and associated neurodevelopmental disorders.” The effectiveness of these dietary supplements and specialized diets is debated because in many cases preliminary studies investigating their efficacy are small in scope and/or follow up investigations have provided conflicting results. However in the vast majority of cases in which a dietary supplement or combination of supplements has made claims regarding its utility in treating ADHD symptoms, no published study exists whatsoever, a sound scientific rationale for its use is absent, and in no case our dietary supplements required to provide evidence of content or purity. In the United States, no dietary supplement has been approved for the treatment for ADHD by the FDA,

EEG biofeedback also sometimes referred to as neurofeedback is purportedly effective in treating attention, impulsivity and hyperactivity. However, the number of patients studied in randomized, placebo-controlled studies is relatively small. Since there are few known side effects from biofeedback therapy it is an attractive alternative although further research is waranted to rigorously assess its effectiveness.

Aerobic fitness may improve cognitive functioning and neural organization related to executive control during pre-adolescent development, though as with biofeedback and other “alternative” treatments, more controlled studies of larger patient populations utilizing standardized rating instruments are needed in this area. One study suggests that athletic performance in boys with ADHD may increase peer acceptance when accompanied by fewer negative behaviors.

People with ADHD tend to work better in less structured environments with fewer rules. Self-employment or jobs with greater autonomy are generally well suited for them. Hyperactive types are likely to change jobs often due to their constant need for new interests and stimulations to keep motivated.

### II. FATIGUE DISORDERS

Isoprophylphenidate is also proposed for treatment of a wide-variety of fatigue disorders, syndromes, or to offset persistent side effects of a number of medications. Some examples of these are listed and discussed below.

A. Drug-Induced Fatigue

Drug-induced fatigue is a common side-effect to a wide variety of drug therapies. Drugs that have been associated with fatigue include antidepressants, such as clomipramine (Anafranil®), isocarboxazid (Marplan®), diazepam (Valium®), alprazolam (Xanax®), prazepam (Centrax®), trazodone (Desyrel®), sertraline (Zoloft®), and buspirone (BuSpar®), as well as antipsychotics.

Another class of drugs that causes fatigue is the anti-hypertensives, including guanadrel (Hylorel®), doxazosin (Cardura®), metoprolol (Lopressor®, Toprol®), hydrochlorothiazide (HCTZ®), acebutolol (Sectral®), atenolol (Tenormin®), tomolol (Blocadren®), atenolol and chlorthalidone (Tenoretic®), carteolol (Cartrol®), clonidine (Catapres®).

Immune agents also cause fatigue, including interferon a (Intron®, Roferon-a®) Zalcitabine (Hivid®), interferon y-lb (Actimmune®), and interleukin-2 (Proleukin®).

Various other fatigue-inducing agents include anti-malarials such as mefloquine (Lariam®), cystitis medicines like Mesna (Mesnex®), antprotozoans such as pentamidine (NebuPent®), biphosphates such as pamidronate (Aredia®), chelates such as succimer (Chemet®), hepatitis B vaccines (Engerix-B®), antiemetics such as metoclopramide (Reglan®), dermatologic agents including isotretinoin (Accutane®), etritinate (Tegison®), anticancer agents like fludarabine (Fludara®) and Nipent (Pentostatin®), muscle relaxants such as dantrolene (Dantrium®), antianemia drugs like erythropoetin (Epogen®, Procrit®) and filgrastim (Neupogen®), and a wide class of opiate compounds and combinations with other medications such as acetaminophen), including opium, thebaine, papaverine, heroin, codeine, levorphanol, meperidine, fentanyl, hydrocodone, hydromorphone, methadone, morphine, oxycodone, propoxyphene, Anexsia®, Darvocet®, Lorcet®, Percocet®, Percodan®, Roxicet®, Tylenol® with Codeine, Tylox®, Vicodin®, Wygesic®, Suboxone and Ziconotide.

B. Sleep Disorders

Sleep disorders also may be treated according to the present invention. Various examples of sleep disorders are discussed below.

1. Narcolepsy

Narcolepsy is a chronic sleep disorder, or dyssomnia. The condition is characterized by excessive daytime sleepiness (EDS) in which a person experiences extreme fatigue and possibly falls asleep at inappropriate times, such as whilst at work or at school. A narcoleptic will most probably experience disturbed nocturnal sleep and also abnormal daytime sleep pattern, which is often confused with insomnia. When a person with narcolepsy falls asleep or goes to bed they will generally experience the 4th stage of sleep REM (rapid eye movement/dreamings state), within 10 minutes; whereas for most people, this shouldn't occur until generally 30 minutes of slumber.

Cataplexy, a sudden muscular weakness brought on by strong emotions (in most cases, there are many people who will experience cataplexy without having a emotional trigger), is know to be one of the other problems that some narcoleptics will experience. Other manifestations include muscular weaknesses ranging from a barely perceptible slackening of the facial muscles to the dropping of the jaw or head, weakness at the knees, or a total collapse. Usually only speech is slurred, vision is impaired (double vision, inability to focus), but hearing and awareness remain normal. In some rare cases, an individual's body becomes paralyzed and muscles will become stiff.

The main characteristic of narcolepsy is excessive daytime sleepiness (EDS), even after adequate night time sleep. A person with narcolepsy is likely to become drowsy or fall asleep, often at inappropriate times and places. Daytime naps may occur with little warning and may be physically irresistible. These naps can occur several times a day. They are typically refreshing, but only for a few hours. Drowsiness may persist for prolonged periods of time. In addition, night time sleep may be fragmented with frequent awakenings.

Four other classic symptoms of the disorder, often referred to as the “tetrad of narcolepsy,” are cataplexy, sleep paralysis, hypnagogic hallucinations, and automatic behavior. These symptoms may not occur in all patients. Cataplexy is an episodic condition featuring loss of muscle function, ranging from slight weakness (such as limpness at the neck or knees, sagging facial muscles, or inability to speak clearly) to complete body collapse. Episodes may be triggered by sudden emotional reactions such as laughter, anger, surprise, or fear, and may last from a few seconds to several minutes. The person remains conscious throughout the episode. In some cases, cataplexy may resemble epileptic seizures. Sleep paralysis is the temporary inability to talk or move when waking (or less often, falling asleep). It may last a few seconds to minutes. This is often frightening but is not dangerous. Hypnagogic hallucinations are vivid, often frightening, dreamlike experiences that occur while dozing, falling asleep and/or while awakening.

Automatic behavior means that a person continues to function (talking, putting things away, etc.) during sleep episodes, but awakens with no memory of performing such activities. It is estimated that up to 40 percent of people with narcolepsy experience automatic behavior during sleep episodes. Sleep paralysis and hypnagogic hallucinations also occur in people who do not have narcolepsy, but more frequently in people who are suffering from extreme lack of sleep. Cataplexy is generally considered to be unique to narcolepsy and is analogous to sleep paralysis in that the usually protective paralysis mechanism occurring during sleep is inappropriately activated. The opposite of this situation (failure to activate this protective paralysis) occurs in rapid eye movement behavior disorder.

In most cases, the first symptom of narcolepsy to appear is excessive and overwhelming daytime sleepiness. The other symptoms may begin alone or in combination months or years after the onset of the daytime naps. There are wide variations in the development, severity, and order of appearance of cataplexy, sleep paralysis, and hypnagogic hallucinations in individuals. Only about 20 to 25 percent of people with narcolepsy experience all four symptoms. The excessive daytime sleepiness generally persists throughout life, but sleep paralysis and hypnagogic hallucinations may not.

Although these are the common symptoms of narcolepsy, many people with narcolepsy also suffer from insomnia for extended periods of time. The symptoms of narcolepsy, especially the excessive daytime sleepiness and cataplexy, often become severe enough to cause serious problems in a person's social, personal, and professional life. Normally, when an individual is awake, brain waves show a regular rhythm. When a person first falls asleep, the brain waves become slower and less regular. This sleep state is called non-rapid eye movement (NREM) sleep. After about an hour and a half of NREM sleep, the brain waves begin to show a more active pattern again. This sleep state, called REM sleep (rapid eye movement sleep), is when most remembered dreaming occurs. Associated with the EEG-observed waves during REM sleep, muscle atonia is present (called REM atonia).

In narcolepsy, the order and length of NREM and REM sleep periods are disturbed, with REM sleep occurring at sleep onset instead of after a period of NREM sleep. Thus, narcolepsy is a disorder in which REM sleep appears at an abnormal time. Also, some of the aspects of REM sleep that normally occur only during sleep - lack of muscular control, sleep paralysis, and vivid dreams - occur at other times in people with narcolepsy. For example, the lack of muscular control can occur during wakefulness in a cataplexy episode; it is said that there is intrusion of REM atonia during wakefulness. Sleep paralysis and vivid dreams can occur while falling asleep or waking up. Simply put, the brain does not pass through the normal stages of dozing and deep sleep but goes directly into (and out of) rapid eye movement (REM) sleep.

This has several consequences. Night time sleep does not include as much deep sleep, so the brain tries to “catch up” during the day, hence EDS. People with narcolepsy may visibly fall asleep at unpredicted moments (such motions as head bobbing are common). People with narcolepsy fall quickly into what appears to be very deep sleep, and they wake up suddenly and can be disoriented when they do (dizziness is a common occurrence). They have very vivid dreams, which they often remember in great detail. People with narcolepsy may dream even when they only fall asleep for a few seconds.

Although the cause of narcolepsy was not determined for many years after its discovery, scientists had discovered conditions that seemed to be associated with an increase in an individual's risk of having the disorder. Specifically, there appeared to be a strong link between narcoleptic individuals and certain genetic conditions. One factor that seemed to predispose an individual to narcolepsy involved an area of Chromosome 6 known as the HLA complex. There appeared to be a correlation between narcoleptic individuals and certain variations in HLA genes, although it was not required for the condition to occur. Certain variations in the HLA complex were thought to increase the risk of an auto-immune response to protein-producing neurons in the brain. The protein produced, called hypocretin or orexin, is responsible for controlling appetite and sleep patterns. Individuals with narcolepsy often have reduced numbers of these protein-producing neurons in their brains. In 2009 the autoimmune hypothesis was supported by research carried out at Stanford University School of Medicine.

The neural control of normal sleep states and the relationship to narcolepsy are only partially understood. In humans, narcoleptic sleep is characterized by a tendency to go abruptly from a waking state to REM sleep with little or no intervening non-REM sleep. The changes in the motor and proprioceptive systems during REM sleep have been studied in both human and animal models. During normal REM sleep, spinal and brainstem alpha motor neuron depolarization produces almost complete atonia of skeletal muscles via an inhibitory descending reticulospinal pathway. Acetylcholine may be one of the neurotransmitters involved in this pathway. In narcolepsy, the reflex inhibition of the motor system seen in cataplexy is believed identical to that seen in normal REM sleep.

In 2004 researchers in Australia induced narcolepsy-like symptoms in mice by injecting them with antibodies from narcoleptic humans. The research has been published in the Lancet providing strong evidence suggesting that some cases of narcolepsy might be caused by autoimmune disease. Narcolepsy is strongly associated with HLA DQB1*0602 genotype. There is also an association with HLA DR2 and HLA DQ1. This may represent linkage disequilibrium. Despite the experimental evidence in human narcolepsy that there may be an inherited basis for at least some forms of narcolepsy, the mode of inheritance remains unknown. Some cases are associated with genetic diseases such as Niemann-Pick disease or Prader-Willi syndrome.

Diagnosis is relatively easy when all the symptoms of narcolepsy are present, but if the sleep attacks are isolated and cataplexy is mild or absent, diagnosis is more difficult. It is also possible for cataplexy to occur in isolation. Two tests that are commonly used in diagnosing narcolepsy are the polysomnogram and the multiple sleep latency test (MSLT). These tests are usually performed by a sleep specialist. The polysomnogram involves continuous recording of sleep brain waves and a number of nerve and muscle functions during nighttime sleep. When tested, people with narcolepsy fall asleep rapidly, enter REM sleep early, and may awaken often during the night. The polysomnogram also helps to detect other possible sleep disorders that could cause daytime sleepiness.

For the multiple sleep latency test, a person is given a chance to sleep every 2 hours during normal wake times. Observations are made of the time taken to reach various stages of sleep (sleep onset latency). This test measures the degree of daytime sleepiness and also detects how soon REM sleep begins. Again, people with narcolepsy fall asleep rapidly and enter REM sleep early.

Treatment is tailored to the individual, based on symptoms and therapeutic response. The time required to achieve optimal control of symptoms is highly variable, and may take several months or longer. Medication adjustments are also frequently necessary, and complete control of symptoms is seldom possible. While oral medications are the mainstay of formal narcolepsy treatment, lifestyle changes are also important.

The primary pharmacological treatments of excessive daytime sleepiness in narcolepsy is with a group of drugs called central nervous system stimulants such as methylphenidate, mixed-amphetamine salts (d- & l-isomers), dextroamphetamine, and methamphetamine, or modafinil (Provigil®), a newer “cognitive enhancing” agent exhibiting a different pharmacologic mechanism of action than tradidional stimulants such as amphetamines. In the Fall of 2007 an alert for severe adverse skin reactions to modafinil was issued by the FDA. Other medications sometimes used are codeine and selegiline. Additionally, the ADHD therapeutic agent atomoxetine (Strattera®), a non-stimulant and NE reuptake inhibitor (NRI),that appears to have little to no abuse potential. In many cases, planned regular short naps can reduce the need for pharmacological treatment of the EDS. Gamma-hydroxybutyrate (GHB), a medication recently approved by the FDA, is the only medication specifically indicated for cataplexy. GHB has also been shown to reduce symptoms of EDS associated with narcolepsy. The precise mechanism of action of GHB is not fully understood. Cataplexy and other REM-sleep symptoms are frequently treated with tricyclic antidepressants such as clomipramine, imipramine, or protriptyline, as well as other drugs that suppress REM sleep. Venlafaxine, a SNRI antidepressant, has shown some usefulness in managing symptoms of cataplexy.

In addition to drug therapy, an important part of treatment is scheduling short naps (10 to 15 minutes) two to three times per day to help control excessive daytime sleepiness and help the person stay as alert as possible. Daytime naps are not a replacement for nighttime sleep. Ongoing communication between the health care provider, patient, and the patient's family members is important for optimal management of narcolepsy. Finally, a recent study reported that transplantation of hypocretin neurons into the pontine reticular formation in rats is feasible, indicating the development of alternative therapeutic strategies in addition to pharmacological interventions.

2. Obstructive Sleep Apnea/Hypopnea Syndrome

Sleep apnea is a sleep disorder characterized by pauses in breathing during sleep. Each episode, called an apnea, lasts long enough so that one or more breaths are missed, and such episodes occur repeatedly throughout sleep. The standard definition of any apneic event includes a minimum 10 second interval between breaths, with either a neurological arousal (a 3-second or greater shift in EEG frequency, measured at C3, C4, O1, or O2), a blood oxygen desaturation of 3-4% or greater, or both arousal and desaturation. Sleep apnea is diagnosed with an overnight sleep test called a polysomnogram.

Clinically significant levels of sleep apnea are defined as five or more episodes per hour of any type of apnea (from the polysomnogram). There are three distinct forms of sleep apnea: central, obstructive, and complex (i.e., a combination of central and obstructive) constituting 0.4%, 84% and 15% of cases respectively. Breathing is interrupted by the lack of respiratory effort in central sleep apnea; in obstructive sleep apnea, breathing is interrupted by a physical block to airflow despite respiratory effort. In complex or “mixed” sleep apnea, there is a transition from central to obstructive features during the events themselves.

Regardless of type, the individual with sleep apnea is rarely aware of having difficulty breathing, even upon awakening. Sleep apnea is recognized as a problem by others witnessing the individual during episodes or is suspected because of its effects on the body. Symptoms may be present for years, or even decades, without identification, during which time the sufferer may become conditioned to the daytime sleepiness and fatigue associated with significant levels of sleep disturbance.

Obstructive sleep apnea (OSA) is the most common category of sleep-disordered breathing. The muscle tone of the body ordinarily relaxes during sleep and at the level of the throat the human airway is composed of collapsible walls of soft tissue which can obstruct breathing during sleep. Mild, occasional sleep apnea, such as many people experience during an upper respiratory infection may not be important, but chronic, severe obstructive sleep apnea requires treatment to prevent low blood oxygen (hypoxemia), sleep deprivation, and other complications. The most serious complication is a severe form of congestive heart failure called cor pulmonale.

Individuals with low muscle tone and soft tissue around the airway (e.g., due to obesity), and structural features that give rise to a narrowed airway are at high risk for obstructive sleep apnea. The elderly are more likely to have OSA than young people. Men are more typical sleep apnea sufferers than women and children, although it is not uncommon in the latter two.

Common symptoms include loud snoring, restless sleep, and sleepiness during the daytime. Diagnostic tests include home oximetry or polysomnography in a sleep clinic.

Some treatments involve lifestyle changes, such as avoiding alcohol or muscle relaxants, losing weight, and quitting smoking Many people benefit from sleeping at a 30 degree elevation of the upper body or higher, as if in a recliner. Doing so helps prevent the gravitational collapse of the airway. Lateral positions (sleeping on a side), as opposed to supine positions (sleeping on the back), are also recommended as a treatment for sleep apnea, largely because the gravitational component is smaller in the lateral position. Some people benefit from various kinds of oral appliances to keep the airway open during sleep. “Breathing machines” like the continuous positive airway pressure (CPAP) may help. There are also surgical procedures to remove and tighten tissue and widen the airway.

As mentioned, snoring is almost a uniform finding in an individual with this syndrome. Snoring is the turbulent sound of air moving through the back of the mouth, nose, and throat. Although not everyone who snores is experiencing difficulty breathing, its presence in combination with other associated symptoms or physiology such as obesity have been found to be highly predictive of OSA risk. The loudness of the snoring is not indicative of the severity of obstruction, however. If the upper airways are tremendously obstructed, there may not be enough air movement to make much sound. Even the loudest snoring does not mean that an individual has sleep apnea syndrome. The sign that is most suggestive of sleep apneas occurs if snoring stops. If it does, along with breath, while the persons' chest and body tries to breathe—that is literally a description of an event in obstructive sleep apnea syndrome. When breathing starts again, there is typically a deep gasp, and then the resumption of snoring.

The term “sleep disordered breathing” is commonly used in the US to describe the full range of breathing problems during sleep in which not enough of air reaches the lungs (hypopnea and apnea). Sleep disordered breathing is associated with an increased risk of cardiovascular disease, stroke, high blood pressure, arrhythmias, diabetes, and accidents. When high blood pressure is caused by OSA, it is distinctive in that, unlike most cases of high blood pressure (so-called essential hypertension), the readings do not drop significantly when the individual is sleeping. Stroke is associated with obstructive sleep apnea. Sleep apnea sufferers also have a 30% higher risk of heart attack or premature death than those unaffected.

Researchers have revealed that people with OSA show tissue loss in brain regions that help store memory, thus linking OSA with memory loss. Using magnetic resonance imaging (MRI), the scientists discovered that sleep apnea patients' mammillary bodies were nearly 20 percent smaller, particularly on the left side. One of the key investigators hypothesized that repeated drops in oxygen lead to the brain injury.

In pure central sleep apnea or Cheyne-Stokes respiration, the brain's respiratory control centers are imbalanced during sleep. Blood levels of carbon dioxide, and the neurological feedback mechanism that monitors it does not react quickly enough to maintain an even respiratory rate, with the entire system cycling between apnea and hyperpnea, even during wakefulness. The sleeper stops breathing, and then starts again. There is no effort made to breathe during the pause in breathing: there are no chest movements and no struggling. After the episode of apnea, breathing may be faster (hyperpnea) for a period of time, a compensatory mechanism to blow off retained waste gases and absorb more oxygen.

While sleeping, a normal individual is at rest, as far as cardiovascular workload is concerned. Breathing is regular in a healthy person during sleep, and oxygen levels and carbon dioxide levels in the bloodstream stay fairly constant. The respiratory drive is so strong that even conscious efforts to hold one's breath do not overcome it. Any sudden drop in oxygen or excess of carbon dioxide (even if tiny) strongly stimulates the brain's respiratory centers to breathe.

In central sleep apnea, the basic neurological controls for breathing rate malfunction and fail to give the signal to inhale, causing the individual to miss one or more cycles of breathing. If the pause in breathing is long enough, the percentage of oxygen in the circulation will drop to a lower than normal level (hypoxaemia) and the concentration of carbon dioxide will build to a higher than normal level (hypercapnia). In turn, these conditions of hypoxia and hypercapnia will trigger additional effects on the body. Brain cells need constant oxygen to live, and, if the level of blood oxygen goes low enough for long enough, the consequences of brain damage and even death will occur. Fortunately, central sleep apnea is more often a chronic condition that causes much milder effects than sudden death. The exact effects of the condition will depend on how severe the apnea is, and the individual characteristics of the person having the apnea.

In any person, hypoxia and hypercapnia have certain common effects on the body. The heart rate will increase, unless there are such severe co-existing problems with the heart muscle itself or the autonomic nervous system that makes this compensatory increase impossible. The more translucent areas of the body will show a bluish or dusky cast from cyanosis, which is the change in hue that occurs due to lack of oxygen in the blood (“turning blue”). Overdoses of drugs that are respiratory depressants (such as heroin, and other opiates) kill by damping the activity of the brain's respiratory control centers. In central sleep apnea, the effects of sleep alone can remove the brain's mandate for the body to breathe. Even in severe cases of central sleep apnea, the effects almost always result in pauses that make breathing irregular, rather than cause the total cessation of breathing.

After exhalation, the blood level of oxygen decreases and that of carbon dioxide increases. Exchange of gases with a lungful of fresh air is necessary to replenish oxygen and rid the bloodstream of built-up carbon dioxide. Oxygen and carbon dioxide receptors in the blood stream (called chemoreceptors) send nerve impulses to the brain, which then signals reflex opening of the larynx (so that the opening between the vocal cords enlarges) and movements of the rib cage muscles and diaphragm. These muscles expand the thorax (chest cavity) so that a partial vacuum is made within the lungs and air rushes in to fill it.

During central apneas, the central respiratory drive is absent, and the brain does not respond to changing blood levels of the respiratory gases. No breath is taken despite the normal signals to inhale. The immediate effects of central sleep apnea on the body depend on how long the failure to breathe endures. At worst, central sleep apnea may cause sudden death. Short of death, drops in blood oxygen may trigger seizures, even in the absence of epilepsy. In people with epilepsy, the hypoxia caused by apnea may trigger seizures that had previously been well controlled by medications. In other words, a seizure disorder may become unstable in the presence of sleep apnea. In adults with coronary artery disease, a severe drop in blood oxygen level can cause angina, arrhythmias, or heart attacks (myocardial infarction). Longstanding recurrent episodes of apnea, over months and years, may cause an increase in carbon dioxide levels that can change the pH of the blood enough to cause a metabolic acidosis.

Any individual, no matter how healthy, who is given enough of a central respiratory depressant drug will develop apnea on a central basis. Generally, drugs that are central respiratory depressants also have sedative effects, and so the individual taking a toxic dose of such a drug is likely to be asleep, or at least in an altered state of consciousness, when breathing becomes irregular. Alcohol is such a central respiratory depressant in large doses, so are opiates, barbiturates, benzodiazepines, and many other tranquilizers. Some individuals have abnormalities that predispose them to central sleep apnea. The treatment for the condition depends on its specific cause.

Similarly, in any person who has some form of sleep apnea (including obstructive sleep apnea), breathing irregularities during sleep can be dangerously aggravated by taking one of these drugs. Quantities that are normally considered safe may cause the person with chronic sleep apnea to stop breathing altogether. Should these individuals have general anesthesia, for example, they require prolonged monitoring after initial recovery, as compared to a person with no history of sleep apnea, because apnea is likely to occur with even low levels of the drugs in their system.

Premature infants with immature brains and reflex systems are at high risk for central sleep apnea syndrome, even if these babies are otherwise healthy. Fortunately, those premature babies who have the syndrome will generally outgrow it as they mature, providing they receive careful enough monitoring and supportive care during infancy to survive. Because of the propensity toward apnea, medications that can cause respiratory drive depression are either not given to premature infants, or given under careful monitoring, with equipment for resuscitation immediately available. Such precautions are routinely taken for premature infants after general anesthesia. Caffeine has been found to help reduce apnea in preterm infants and to aid in care after general anesthesia.

Adults suffering from congestive heart failure are at risk for a form of central sleep apnea called Cheyne-Stokes respiration. This is periodic breathing with recurrent episodes of apnea alternating with episodes of rapid breathing. In those who have it, Cheyne-Stokes respirations occur while both awake and asleep. There is good evidence that replacement of the failed heart (heart transplant) cures central apnea in these patients. The use of some medications that are respiratory stimulants decrease the severity of apnea in some patients.

3. Shift Work Sleep Disorder

Shift Work Sleep Disorder (SWSD) is a circadian rhythm sleep disorder which affects people who change their work or sleep schedules frequently or work longterm on other than the day shift. Schedules of these people go against the body's natural Circadian rhythm, and individuals have difficulty adjusting to the different sleep and wake schedule. SWSD consists of a constant or recurrent pattern of sleep interruption that results in insomnia or excessive sleepiness. This disorder is common in people who work non-traditional hours, usually between 10:00 p.m. and 6:00 a.m. Both the DSM-IV-TR (Text Revision, 2000) and the International Statistical Classification of Diseases and Related Health Problems 10th Revision or (ICD-10) both recognize SWSD as a coded disorder falling under the headings of Circadian Rhythm Sleep Disorder and Disorders of The Sleep-Wake Schedule, respectively. Common symptoms of SWSD are insomnia and excessive sleepiness, and may also include difficulty concentrating, headaches, and lack of energy. Shift Work Sleep Disorder can lead to increased accidents, increased work-related errors, increased sick leave and increased irritability and mood problems.

Shift workers must make sleep a priority, even though it might be daylight outside. One should follow bedtime rituals and try to keep a regular sleep schedule - even on weekends, go to sleep as soon as possible after work, and try to get at least 7 to 8 hours of sleep every day. Works should also decrease the number of night shifts worked in a row, avoid extended work hours, avoid long commutes, avoid frequently rotating shifts and get enough sleep on days off

C. Sleep-Depriving Diseases

There are also a number of disease that can cause fatigue, and which thus can be treated with isopropylphentidate in accordance with the present invention.

1. Depression

Major depressive disorder, also known as clinical depression, major depression, unipolar depression, or unipolar disorder, is a mental disorder characterized by an all-encompassing low mood accompanied by low self-esteem, and loss of interest or pleasure in normally enjoyable activities. The term “major depressive disorder” was selected by the American Psychiatric Association to designate this symptom cluster as a mood disorder in the (DSM-III, 1980), and has become widely used since. The general term depression is often used to describe the disorder, but as it can also be used to describe other types of psychological depression, more precise terminology is preferred for the disorder in clinical and research use. Major depression is a disabling condition which adversely affects a person's family, work or school life, sleeping and eating habits, and general health. In the United States, approximately 3.4% of people with major depression commit suicide, and up to 60% of people who commit suicide have depression or another mood disorder.

The diagnosis of major depressive disorder is based on the patient's self-reported experiences, behavior reported by relatives or friends, and a mental status exam. There is no laboratory test for major depression, although physicians generally request tests for physical conditions that may cause similar symptoms. The most common time of onset is between the ages of 30 and 40 years, with a later peak between 50 and 60 years. Major depression is reported about twice as frequently in women as in men, although men are at higher risk for committing suicide.

Most patients are treated in the community with antidepressant medication and some with psychotherapy or counseling. Hospitalization may be necessary in cases with associated self-neglect or a significant risk of harm to self or others. A minority are treated with electroconvulsive therapy (ECT), under a short-acting general anesthetic. The course of the disorder varies widely, from one episode lasting months to a lifelong disorder with recurrent major depressive episodes. Depressed individuals have shorter life expectancies than those without depression, in part because of greater susceptibility to medical illnesses. Current and former patients may be stigmatized.

The understanding of the nature and causes of depression has evolved over the centuries, though many aspects of depression remain incompletely understood and are the subject of discussion and research. Psychological, psycho-social, hereditary, evolutionary and biological causes have been proposed. Psychological treatments are based on theories of personality, interpersonal communication, and learning. Most biological theories focus on the CNS monoamines 5-HT, NE, and DA that are naturally present in the brain and assist communication between nerve cells. Monoamines have been implicated in depression, and most antidepressants work to increase the active levels of at least one.

Major depression is a serious illness that affects a person's family, work or school life, sleeping and eating habits, and general health. Its impact on functioning and well-being has been equated to that of chronic medical conditions such as diabetes.

An individual suffering from a major depressive episode typically exhibits a very low mood that pervades all aspects of life and an inability to experience pleasure in activities that formerly were enjoyed. Depressed individuals may be preoccupied with, or ruminate over, thoughts and feelings of worthlessness, inappropriate guilt or regret, helplessness, hopelessness, and self-hatred. Other symptoms include poor concentration and memory, withdrawal from social situations and activities, reduced sex drive, and thoughts of death or suicide. Insomnia is common: in the typical pattern, a person wakes very early and is unable to get back to sleep. Hypersomnia, or oversleeping, is less common. Appetite often decreases, with resulting weight loss, although increased appetite and weight gain occasionally occur. The person may report multiple physical symptoms such as fatigue, headaches, or digestive problems; physical complaints are the most common presenting problem in developing countries according to the World Health Organization's criteria of depression. Family and friends may notice that the person's behavior is either agitated or lethargic.

Older depressed persons may have cognitive symptoms of recent onset, such as forgetfulness, and a more noticeable slowing of movements. Depression often coexists with physical disorders common among the elderly, such as stroke, other cardiovascular diseases, Parkinson's disease, and chronic obstructive pulmonary disease.

In very severe cases, depressed individuals may have symptoms of psychosis such as delusions or, less commonly, hallucinations, usually of an unpleasant nature.

Depressed children often display an irritable rather than a depressed mood, and show varying symptoms depending on age and situation. Most exhibit a loss of interest in school and a decline in academic performance. They may be described as clingy, demanding, dependent, or insecure. Diagnosis may be delayed or missed when symptoms are interpreted as normal moodiness. Depression may also coincide with attention-deficit hyperactivity disorder, complicating the diagnosis and treatment of both.

The biopsychosocial model proposes that biological, psychological, and social factors all play a role to varying degrees in causing depression. The diathesis—stress model posits that depression results when a preexisting vulnerability, or diathesis, is activated by stressful life events. The preexisting vulnerability can be either genetic, implying an interaction between nature and nurture, or schematic, resulting from views of the world learned in childhood. These interactive models have gained empirical support. For example, researchers in New Zealand took a prospective approach to studying depression, by documenting over time how depression emerged among an initially normal cohort of people. The researchers concluded that variation among the serotonin transporter (5-HTT) gene affects the chances that people who have dealt with very stressful life events will go on to experience depression. Specifically, depression may follow such events, but seems more likely to appear in people with one or two short alleles of the 5-HTT gene.

A Swedish study estimated the heritability of depression—the degree to which individual differences in occurrence are associated with genetic differences—to be approximately 40% for women and 30% for men, and evolutionary psychologists have proposed that the genetic basis for depression lies deep in the history of naturally selected adaptations. A substance-induced mood disorder resembling major depression has been causally linked to long-term drug use or abuse, or to withdrawal from certain sedative and hypnotic drugs.

The three most common treatments for depression are psychotherapy, medication, and electroconvulsive therapy. Psychotherapy is the treatment of choice for people under 18, while electroconvulsive therapy is only used as a last resort. Care is usually given on an outpatient basis, while treatment in an inpatient unit is considered if there is a significant risk to self or others. A significant number of recent studies have indicated that physical exercise has beneficial effects.

Treatment options are much more limited in developing countries, where access to mental health staff, medication, and psychotherapy is often difficult. Development of mental health services is minimal in many countries; depression is viewed as a phenomenon of the developed world despite evidence to the contrary, and not as an inherently life-threatening condition.

Psychotherapy can be delivered, to individuals or groups, by mental health professionals, including psychotherapists, psychiatrists, psychologists, clinical social workers, counselors, and psychiatric nurses. With more complex and chronic forms of depression, a combination of medication and psychotherapy may be used. In children and young people under 18, medication should only be offered in conjunction with a psychological therapy, such as cognitive behavioral therapy (CBT), interpersonal therapy, or family therapy. Psychotherapy has been shown to be effective in older people. Successful psychotherapy appears to reduce the recurrence of depression even after it has been terminated or replaced by occasional booster sessions.

The most studied form of psychotherapy for depression is CBT, thought to work by teaching clients to learn a set of useful cognitive and behavioral skills. Earlier research suggested that CBT was not as effective as antidepressant medication; however, research in 1996 suggests that it can perform as well as antidepressants in patients with moderate to severe depression. Overall, evidence shows CBT to be effective in depressed adolescents, although one systematic review noted there was insufficient evidence regarding severe episodes. Combining fluoxetine with CBT appeared to bring no additional benefit, or, at the most, only marginal benefit. Several variables predict success for cognitive behavior therapy in adolescents: higher levels of rational thoughts, less hopelessness, fewer negative thoughts, and fewer cognitive distortions.

Several variants of cognitive behavior therapy have been used in depressed patients, most notably rational emotive behavior therapy, and more recently mindfulness-based cognitive therapy.

Interpersonal psychotherapy focuses on the social and interpersonal triggers that may cause depression. The therapy takes a structured course with a set number of weekly sessions (often 12) that focus on relationships with others. Therapy can be used to foster interpersonal skills that allow people to communicate more effectively and to reduce stress.

The effects of prescription antidepressants can be comparable to those of psychotherapy, although more patients cease medication than cease psychotherapy, most likely due to side effects from the medication.

To find the most effective antidepressant medication with tolerable or fewest side effects, the dosages can be adjusted, and if necessary, combinations of different classes of antidepressants can be tried. Response rates to the first antidepressant administered range from 50-75%, and it can take at least six to eight weeks from the start of medication to remission, when the patient is back to their normal self Antidepressant medication treatment is usually continued for 16 to 20 weeks after remission, to minimize the chance of recurrence. People with chronic depression may need to take medication indefinitely to avoid relapse.

Selective serotonin reuptake inhibitors (SSRIs), such as sertraline, escitalopram, fluoxetine, paroxetine, and citalopram are the primary medications prescribed owing to their effectiveness, relatively mild side effects, and because they are less toxic in overdose than other antidepressants. Patients who do not respond to one SSRI can be switched to another, and this results in improvement in almost 50% of cases. Another option is to switch to the atypical antidepressant bupropion. Venlafaxine, an SNRI antidepressant, may be modestly more effective than SSRIs. However, venlafaxine is not recommended in the UK as a first-line treatment because of evidence suggesting its risks may outweigh benefits, and it is specifically discouraged in children and adolescents. For adolescent depression, fluoxetine and escitalopram are the two recommended choices. Antidepressants have not been found to be beneficial in children. Any antidepressant can cause low serum sodium levels (also called hyponatremia); nevertheless, it has been reported more often with SSRIs. It is not uncommon for SSRIs to cause or worsen insomnia; the sedating antidepressant mirtazapine can be used in such cases.

Monoamine oxidase inhibitors (MAOIs), an older class of antidepressants, have fallen out of favor with clinicians given their potentially life-threatening side effect of severe hypertension if specific dietary restrictions and specific drug a voidance is not maintained by the patient. They are still used, but only rarely relative to the newer agents. However, some newer and better-tolerated agents of the MAO class have recently been developed.

The terms refractory depression or treatment-resistant depression are used to describe cases that do not respond to adequate courses of least two antidepressants. In many major studies, only about 35% of patients respond well to medical treatment. It may be difficult for a doctor to decide when someone has treatment-resistant depression or whether the problem is due to coexisting disorders, which are common among patients with major depression.

A doctor may add a medication with a different mode of action to bolster the effect of an antidepressant in cases of treatment resistance. Medication with lithium salts has been used to augment antidepressant therapy in those who have failed to respond to antidepressants alone. Furthermore, lithium dramatically decreases the suicide risk in recurrent depression. Addition of a thyroid hormone, triiodothyronine may work as well as lithium, even in patients with normal thyroid function. Addition of atypical antipsychotics when the patient has not responded to an antidepressant is also known to increase the effectiveness of antidepressant drugs, albeit offset by increased side effects.

Electroconvulsive therapy (ECT) is a procedure whereby pulses of electricity are sent through the brain via two electrodes, usually one on each temple, to induce a seizure while the patient is under a short general anesthetic. Hospital psychiatrists may recommend ECT for cases of severe major depression that have not responded to antidepressant medication or, less often, psychotherapy or supportive interventions. ECT can have a quicker effect than antidepressant therapy and thus may be the treatment of choice in emergencies such as catatonic depression where the patient has stopped eating and drinking, or where a patient is severely suicidal. ECT is probably more effective than pharmacotherapy for depression in the immediate short-term, although a landmark community-based study found much lower remission rates in routine practice. Used on its own the relapse rate within the first six months is very high; early studies put the rate at around 50%, while a more recent controlled trial found rates of 84% even with placebos. The early relapse rate may be reduced by the use of psychiatric medications or further ECT (although the latter is not recommended by some authorities) but remains high. Common initial adverse effects from ECT include short and long-term memory loss, disorientation and headache. Although objective psychological testing shows memory disturbance after ECT has mostly resolved by one month post treatment, ECT remains a controversial treatment, and debate on the extent of cognitive effects and safety continues.

Physical exercise is recommended by some health authorities, and a systematic review of 23 studies indicated a “large clinical effect.” Among these, three studies employing intention to treat analysis and other bias-reducing measures were inconclusive.

St. John's wort is available over-the-counter as a herbal remedy in many parts of the world; however, the evidence of its effectiveness for the treatment of major depression is poor and somewhat equivocal in the treatment of minor to moderate depressive symptoms. Further, its safety may be compromised by inconsistency in pharmaceutical quality and in the amounts of active ingredient in different preparations as there is no requirement for quality control or regulatory guidance in many countries including the United States. Further, components within St. John's wort have been shown to interact with numerous prescribed medicines including antidepressants, and it can reduce the effectiveness of anxiolytics, immunosuppressants, oral contraceptives, and others.

The issue of efficacy of omega-3 fatty acids for major depression is controversial and unresolved, with controlled studies and meta-analyses supporting both positive and negative conclusions. Recent meta-analyses support their use as adjunctive treatments in major depressive disorder. Because side effects are minimal, they may prove a useful approach for pregnant women or children.

Reviews of short-term clinical trials of S-adenosylmethionine (SAMe) indicate that it may be effective in treating major depression in adults. A 2002 review reported that tryptophan and 5-hydroxytryptophan appear to be better than placebo, but it did not recommend their widespread use owing to lack of conclusive evidence on efficacy and safety, and generally preferred the use of safer antidepressants instead.

Repetitive transcranial magnetic stimulation (rTMS) applies powerful magnetic fields to the brain from outside the head. Multiple controlled studies support the use of this method in treatment-resistant depression; it has been approved for this indication in Europe, Canada, Australia, and the US. rTMS appeared similarly effective for both uncomplicated depression and depression resistant to medication; however, it was inferior to ECT in a side-by-side randomized trial.

Vagus nerve stimulation (VNS) was approved by the FDA in the United States in 2005 for use in treatment-resistant depression, although it failed to show short-term benefit in the only large double-blind trial when used as an adjunct on treatment-resistant patients; a 2008 systematic review concluded that despite the promising results reported mainly in open studies, further clinical trials are needed to confirm its efficacy in major depression and the robustness of its effects are in question.

Poor diet or medical disorders leading to deficiency in certain nutrients have been linked to major depression disorder. Thus improved diet or correction of nutritional deficiency may be of value in some cases of major depression.

2. Apathy

Apathy, also called impassivity or perfunctoriness, is a state of indifference, or the suppression of emotions such as concern, excitement, motivation and passion. An apathetic individual has an absence of interest or concern to emotional, social, or physical life. They may also exhibit an insensibility or sluggishness.

Often, apathy has been felt after witnessing horrific acts, such as the killing or maiming of people during a war. It is also known to be associated with many conditions, some of which are: depression, Alzheimer's disease, Chagas' disease, Creutzfeldt-Jakob disease, dementia, Korsakoff's Syndrome, excessive vitamin D, general fatigue, Huntington's disease, Pick's disease, progressive supranuclear palsy (PSP), schizophrenia, Schizoid Personality Disorder, Bipolar Disorder and others. Some medications and the heavy use of drugs such as heroin may bring apathy as a side effect.

### III. ISOPROPYLPHENIDATE, STEREOISOMERS AND SALTS THEREOF

Isoprophylphenidate is an ester homolog of methylphenidate. Its structure, along with that of methylphenidate, are provided in FIG. 1. It can be prepared as a salt, such as, but not limited to hydrochloride, hydrobromide, sulfate tartrate, succinate, aspartate salts.

Isoprophylphenidate can be synthesized as follows: (±)-Ritalinic acid (2 mmol) is dissolved in isopropyl alcohol saturated with HCl gas (75 ml) and refluxed for 24 h under nitrogen. The solution is evaporated to dryness under reduced pressure, purged with nitrogen, and the white residue was then dissolved in a minimum volume of warm isopropyl alcohol. Diethyl ether is then added to turbidity, and the flask was stored for 24 h at 2° C. The resulting white crystalline product is filtered, washed with diethyl ether, and dried under vacuum. The purity of the synthetic material is confirmed by gas chromatography-mass spectrometry.

Methods for resolving stereoisomers of methylphenidate are disclosed in Patrick et al. (1987) and U.S. Pat. No. 6,242,464. Those methods can be applied to the separation of isopropylphenidate stereoisomers as well in view of the prominent activity of the d-isomer (i.e., RR—) configuration in the “phenidate” series of cogeners (e.g., see Patrick et al., 1987). In addition to the “threo” diastereomers (d-threo [RR-], l-threo [SS—] and dl-threo [RR—/SS—]) of isoprophyphenidate, the use of “erythro” diastereomers (d-erythro, l-erythro and dl-erythro) also is contemplated.

IV. THERAPIES

In the context of the present invention, it is contemplated that isopropylphenidate, or salts thereof, may be used either as a monotherapy or in combination with a second therapeutic agent to more effectively treat one or more of the conditions or disorders set forth above. Additional therapeutic agents contemplated for use in combination with isopropylphenidate or salts thereof include any of the drugs or therapies mentioned in preceding sections.

A. Isopropylphenidate Monotherapy

1. Formulations and Routes for Administration

Where clinical applications are contemplated, it will be necessary to prepare pharmaceutical compositions of isopropylphenidate or salts thereof (and additional therapeutic agent disclosed herein) in a form appropriate for the intended application. Generally, this will entail preparing compositions that are essentially free of pyrogens, as well as other impurities that could be harmful to humans or animals. Formulations similar to those used with methylphenidate are particularly contemplated.

One will generally desire to employ appropriate salts and buffers to render delivery agents stable and allow for uptake. Buffers also will be employed when preparing formulations for administration to a patient. Aqueous compositions of the present invention in an effective amount may be dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The composition(s) of the present invention may be delivered orally, sublingually, intranasally, intramuscularly, intraperitoneally, intravenously, and transdermally. In some embodiments, local or regional delivery of isopropylphenidate or salts thereof (alone, or in combination with a second therapeutic agent, as discussed below), to a patient will be a very efficient method of delivery to counteract the disease or disorder.

Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The therapeutic compositions also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH, exact concentration of the various components, and the pharmaceutical composition are adjusted according to well known parameters. Suitable excipients for formulation with isopropylphenidate or salts thereof include croscarmellose sodium, hydroxypropyl methylcellulose, iron oxides synthetic), magnesium stearate, microcrystalline cellulose, polyethylene glycol 400, polysorbate 80, povidone, silicon dioxide, titanium dioxide, and water (purified).

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent(s) of the present invention is determined based on the intended goal and the patient condition. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired, including age, body weight, general health, sex, diet, time of administration, route of administration, rate of excretion, drug combination, and the severity of the particular disease undergoing therapy. Precise amounts of the therapeutic composition also depend on the judgment of the practitioner and are peculiar to each individual.

2. Therapeutically Effective Amounts of Isopropylphenidate

A therapeutically effective amount of isopropylphenidate or salts thereof alone, or when used in combination with a second therapeutic agent, varies depending upon the host treated and the particular mode of administration. In one embodiment of the invention the dose range of the isopropylphenidate or salts thereof alone, or in combination with a second agent used will be in the range of 0.05-2 mg/kg, e.g., about 0.3 mg/kg body weight. The term “body weight” is applicable when a human is being treated. All concentrations and treatment levels are expressed as “body weight” or simply “kg” in this application are also considered to cover the analogous “total cell weight” and “total weight” concentrations. However, those of skill will recognize the utility of a variety of dosage range, for example, 1 mg/kg body weight to 450 mg/kg body weight, 2 mg/kg body weight to 400 mg/kg body weight, 3 mg/kg body weight to 350 mg/kg body weight, 4 mg/kg body weight to 300 mg/kg body weight, 5 mg/kg body weight to 250 mg/kg body weight, 6 mg/kg body weight to 200 mg/kg body weight, 7 mg/kg body weight to 150 mg/kg body weight, 8 mg/kg body weight to 100 mg/kg body weight, or 9 mg/kg body weight to 50 mg/kg body weight. Further, those of skill will recognize that a variety of different dosage levels will be of use, for example, 1 mg/kg, 2 mg/kg, 3 mg/kg, 4 mg/kg, 5 mg/kg, 7.5 mg/kg, 10 mg/kg, 12.5 mg/kg, 15 mg/kg, 17.5 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 120 mg/kg, 140 mg/kg, 150 mg/kg, 160 mg/kg, 180 mg/kg, 200 mg/kg, 225 mg/kg, 250 mg/kg, 275 mg/kg, 300 mg/kg, 325 mg/kg, 350 mg/kg, 375 mg/kg, 400 mg/kg, 450 mg/kg, 500 mg/kg, 550 mg/kg, 600 mg/kg, 700 mg/kg, 750 mg/kg, 800 mg/kg, 900 mg/kg, 1000 mg/kg, 1250 mg/kg, 1500 mg/kg, 1750 mg/kg, 2000 mg/kg, 2500 mg/kg, and/or 3000 mg/kg. Of course, all of these dosages are exemplary, and any dosage in-between these points is also expected to be of use in the invention. Any of the above dosage ranges or dosage levels may be employed for isopropylphenidate or salts thereof alone, or in combination with a second therapeutic agent.

“Therapeutically effective amounts” are those amounts effective to produce beneficial results in the recipient animal or patient. Such amounts may be initially determined by reviewing and comparing the function to MPH in the published literature, by conducting in vitro tests, or by conducting metabolic studies in experimental animals. Preferred animal models for use in certain embodiments are rodent models, which are preferred because they are economical to use and, particularly, because the results gained are widely accepted as predictive of clinical value.

In some embodiments of the present invention therapy may be administered in regular cycles. A cycle may involve one dose, after which several days or weeks without treatment, or during which a second therapy is administered. Doses may be given several days in a row, or every other day for several days, followed by a period of rest. If more than one drug is used (see below), the treatment plan will specify how often and exactly when each drug should be given. The number of cycles a person receives may be determined before treatment starts or may be flexible, in order to take into account how the patient responds. Certain side effects may also require doctors to adjust therapy plans.

B. Combination Therapies

In combination therapies, one would generally contact a cell with isopropylphenidate or salts thereof at the same time. This may be achieved by contacting the cell with a single composition or pharmacological formulation that includes both agents, or by contacting the cell with two distinct compositions or formulations, at the same time, wherein one composition includes isopropylphenidate or salts thereof and the other includes the second agent. It is conceivable that isopropylphenidate-methylphenidate mixtures of various ratios could be designed to provide a dosage form of varying time course of effects based upon the different rates of hCES1-mediated hydrolysis (inactivation) of the respective molecules.

Alternatively, treatment with isopropylphenidate or salts thereof may precede and/or follow the additional treatment by intervals ranging from minutes to weeks. In embodiments where the second agent is applied separately to the cell, one would generally ensure that a significant period of time did not expire between the time of each delivery, such that the agent would still be able to exert an advantageously combined effect on the cell. In such instances, it is contemplated that one would contact the cell with both modalities within about 12-24 hr of each other and, more preferably, within about 6-12 hr of each other, with a delay time of only about 12 hr being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either isopropylphenidate or salts thereof in combination with a second therapeutic agent will be used. Various combinations may be employed, where isopropylphenidate or salts thereof is “A” and the second therapy is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B
B/B/B/A A/A/A/B B/A/A/A A/B/A/A A/A/B/A A/B/B/B
B/A/B/B B/B/A/B

Other combinations are contemplated.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Chemical compounds. dl-threo-methylphenidate (MPH) was purchased from Sigma-Aldrich (St. Louis, Mo.). dl-isopropylphenidate (IPH) was also synthesized in the inventors' laboratory using the following method. In brief, (±)-ritalinic acid (2 mmol) was dissolved in isopropyl alcohol saturated with HCl gas (75 ml) and refluxed for 24 h under nitrogen. The solution was evaporated to dryness under reduced pressure, purged with nitrogen, and the white residue was then dissolved in a minimum volume of warm isopropyl alcohol. Diethyl ether was then added to turbidity, and the flask was stored for 24 h at 2° C. The resulting white crystalline product was filtered, washed with diethyl ether, and dried under vacuum. The purity of the synthetic material was confirmed by gas chromatography-mass spectrometry. All compounds studied were assessed as their HCl salts. The remaining reagents and solvents were of the highest grade commercially available. Animals used in these experiments were male rats of the Sprague-Dawley strain weighing 200-300g and obtained from Charles River Laboratories (Wilmington, Mass.). All chemicals and reagents were from Sigma Chemical Company (St. Louis, Mo.).

Transporter Binding and Cellular Uptake. All described monoamine transporter binding studies as well as cellular uptake assays were performed in duplicate by CEREP (Celle l'Evescault, France) and are described below. Further details of all assays performed may be accessed at the CEREP web site (world-wide-web at cerep.com). Cellular-based assays were conducted to provide complementary in vitro functional measures to complement the standard transporter assays as well as to provide direct comparisons of the homologs IPH with MPH with regard to DA, NE, and 5-HT uptake in synaptosomal preparations. The concentration of MPH, EPH, and IPH utilized in these studies was 10 μM, which represents a generally accepted screening concentration when evaluating compounds in this manner.

Dopamine Transporter Binding Assay. Evaluation of the affinity of both compounds for the human dopamine (DA) transporter (DAT) in transfected Chinese Hamster Ovary (CHO) cells was determined in a validated radioligand binding assay. Cell membrane homogenates were incubated for 120 min at 4° C. with 0.5 nM [³H]GBR 12935 in the absence or presence of each test compound in a standard buffer solution. Nonspecific binding was determined in the presence of 10 μM N-[1-(Benzo[b]thien-2-yl-cyclohexyl)] piperidine (BTCP). Following incubation, the samples were filtered under vacuum through glass filters and rinsed several times with ice-cold 50 mM Tris-HCl using a 96-sample cell harvester. The filters were then dried and measured for radioactivity with a scintillation counter (TopCount, Packard) using a liquid scintillation cocktail (Microscint 0, Packard). The results of DAT binding experiments as well as the other transporter assays described in the following sections were expressed as a percent inhibition of the control radioligand specific binding. The standard reference compound was BTCP, which was tested in each experiment at several concentrations in order to obtain a competition curve from which its IC₅₀ was calculated.

Norepinephrine Transporter Binding Assay. Evaluation of the affinity of the two compounds for the human norepinephrine (NE) transporter (NET) in transfected CHO cells was determined in a radioligand assay analogous to the procedures described for the DAT assay. Cell membrane homogenates were incubated for 90 min at 4° C. with 1 nM [³H]nisoxetine in the absence or presence of each test compound in a standard buffer solution. Nonspecific binding was determined in the presence of 1 μM of desipramine. Following incubation, the samples were filtered rapidly under vacuum and rinsed several times with a buffer solution. The filters were then dried and radioactivity counts were obtained. The results are expressed as percent inhibition of the control radioligand specific binding. The standard reference compound was the tricyclic antidepressant (TCA) protriptyline, which was tested in each experiment at several concentrations to generate a competition curve from which its IC₅₀ was calculated.

Serotonin Transporter Binding Assay. An evaluation of the respective compounds was also carried out regarding their affinity for the human serotonin 5-HT transporter (SERT) in transfected CHO cells. Briefly, cell membrane homogenates were incubated for 90 min at 4° C. with 2 nM of [³H]imipramine in the absence and presence of each of the assessed compounds. Nonspecific binding was determined in the presence of 10 μM of imipramine. After incubation, the samples were filtered rapidly under vacuum and rinsed several times with buffer solution. The filters were then dried and measured for radioactivity via scintillation counter. The standard reference compound was the TCA imipramine, which was tested in each experiment at several concentrations to generate a competition curve from which its IC₅₀ was calculated.

Norepinephrine Uptake. The evaluation of the effects of each compound of interest (MPH, IPH) on NE uptake utilized synaptosomes prepared from the rat hypothalamus. These synaptosomes (100 μg) were incubated for 20 min at 37° C. with 0.1 μCi [³H]norepinephrine in the absence (i.e., control) or presence of the test compound or the reference compound in a standard buffer solution. Basal control activity was determined by incubating the same mixture for 20 min at 0° C. in the presence of 10 μM protriptyline to block the uptake. Following incubation, the samples were filtered, counted using a scintillation instrument, and the results expressed as a percent inhibition of the control uptake of [³H]norepinephrine. The standard inhibitory reference compound was the TCA protriptyline, which was tested in each experiment at several concentrations to obtain an inhibition curve from which its IC₅₀ value was calculated.

Dopamine Uptake. The evaluation of the effects of the two compounds on DA uptake again utilized synaptosomes but this time prepared from the rat striatum. The synaptosomes were incubated for 15 min at 37° C. with 0.1 μCi [³H]dopamine in the absence and presence of the test compound or the reference compound in a buffer standard buffer solution. Basal control activity was determined by incubating the same mixture for 15 min at 4° C. in the presence of 1 μM GBR12909 to block the uptake. After incubation, the samples were filtered, counted, and the results expressed as a percent inhibition of the control uptake of [³H]dopamine by scintillation count. The standard inhibitory reference compound was GBR12909, which was tested in each experiment at several concentrations to obtain an inhibition curve from which its IC₅₀ value was calculated.

Serotonin Uptake. The assessment of the relative effects of IPH and MPH on 5-HT uptake utilized measures of [³H]5-HT incorporation into synaptosomes prepared from the rat brain. The synaptosomes were incubated for 15 min at 37° C. with [³H]5-HT (0.2 μCi/ml) in the absence and presence of each of the two assessed compounds or the reference compounds. Following incubation, the samples were filtered, counted using a scintillation instrument, and the results expressed as a percent inhibition of the control uptake of [³H]5-HT. The standard inhibitory reference compound was the TCA imipramine, which was tested in each experiment at several concentrations to obtain an inhibition curve from which its IC₅₀ value was calculated.

Determination of Relative Hydrolytic Rates of MPH and IPH. The assessment of relative rates of hydrolysis of the two ester compounds utilized both cell culture and human liver microsomes (HLM) and human intestinal microsomes (HIM). The human carboxylesterase 1 (hCES1) s9 fraction was prepared utilizing a novel method developed in the inventors' laboratory previously (Zhu et al., 2008). Hydrolysis of IPH and MPH by hCES1, HLM, and human intestinal microsomes was assessed by incubating the substrates with the respective enzymes and measuring the formation of the major metabolite RA formed via ester hydrolysis. Briefly, 50 μl of freshly prepared substrates (IPH and MPH) was mixed with 50 μl of enzymes in 1.5 ml Eppendorf tubes yielding a total volume of 100 μl. The final substrate concentration was 1 mM and the final enzyme concentrations are 0.5 mg/ml, 0.2 mg/ml, 0.2 mg/ml for hCES1, HLM, and HIM, respectively. Following incubation at 37° C. for 60 min, the reaction was terminated by adding 500 μl of methanol. Precipitated protein was then removed by centrifugation (20,000g for 5 min at 4° C.). Concentrations of the primary hydrolytic product of the two compounds, RA were determined utilizing a validated HPLC method previously described.

An In Vitro Investigation of the Transesterification Potential of Isopropylphenidate versus Methylphenidate in the Presence of Ethanol. The s9 fraction of hCES1-transfected cells, d1-MPH), dl-IPH, and ethanol working solution were prepared in DPBS containing 20 mM HEPES, pH7.4. The reaction was initiated by mixing 200 μl of s9 fraction, 100 μl of substrates (MPH or IPH) and 100 μl of ethanol. The final concentrations of s9 fraction protein, MPH, IPH, and ethanol were 2 mg/ml, 1 mM, 1 mM, and 10 mM, respectively. After incubation at 37° C. for 1 h, the reaction was terminated by adding a four-fold concentration of ice-cold methanol. The samples were then centrifuged at 16,000 g for 5 min, and the resulting supernatant was subjected to HPLC analysis to assess each for the formation and/or concentration of the hydrolytic product of both MPH and IPH, and common metabolite RA. Additionally, the samples were assayed for the presence of the transesterification metabolite ethylphenidate (EPH). Further, the s9 fraction prepared from the vector-transfected cells was included in this study as a control for any non-enzymatic hydrolysis which might occur. The hCES1 mediated MPH and IPH hydrolysis was estimated by deducting the amounts of RA produced in the vector s9 samples from the RA formation observed in the hCES1 s9 samples.

Locomotor Activity Measurement in Rats. Locomotor-inducing activity of IPH was measured according to methods previously described by Patrick and associates during their establishment the differential pharmacology of the enantiomers of MPH (Patrick et al., 1987) as well as hydroxylated metabolites of MPH (Patrick et al., 1981). The present study compared only IPH homolog versus saline dosing since SMPH has been extensively investigated in vivo previously. In brief, following an initial 60 min habituation period to the activity chamber, racemic IPH (or saline) was administered intraperitonealy (i.p.) to each animal (n=5 per active drug group; n=3 per saline group) at a dose of 10 mg/kg to correspond with 10 mg of racemic MPH (delivering 5 mg of d-MPH) which was previously determined to produce maximal behavioral responses (Patrick et al., 1987). Additionally, the MPH analog d-EPH was also recently shown to produce significant motor activity in a mouse model following i.p. dosing at 5 mg/kg (Patrick et al., 2005). Locomotor activity was recorded within doughnut-shaped cages with six photocell sensors equally spaced around a 9 cm runway. Activity counts as assessed by light-beam interruptions were recorded for each animal in increments of 10 minutes over a 2 hr period. Differences in the cumulative motor activity accounts were assessed by the unpaired, two-tailed Student t-test. The level of significance was set at p<0.05.

### Example 2

### Results

Monoamine Transporter Binding and Cellular Uptake Studies. The binding of IPH and MPH to the prominent cellular monoamine transporters DAT, NET, and SERT revealed that binding affinity was greatest for DAT. Both compounds produced significant effects at the DAT with insignificant differences noted between the two compounds (Table 1). With regard to NET, as anticipated, MPH exhibited substantial binding as measured by inhibition of specific control binding while IPH exhibited the lowest affinity at a value approximating one-third that of MPH. In the case of SERT, neither of the compounds assessed either approached or exceeded 50% inhibition of control specific binding.

The results of monoamine cellular (i.e., functional) assays are presented in Table 2. With regard to DA, both compounds exhibited significant effects on the uptake of this monoamine with little difference observed between the agents. Norepinephrine uptake studies indicated that MPH exerted significant effects while IPH was much lower. Finally, 5-HT uptake was not affected to any significant degree by either assessed compound.

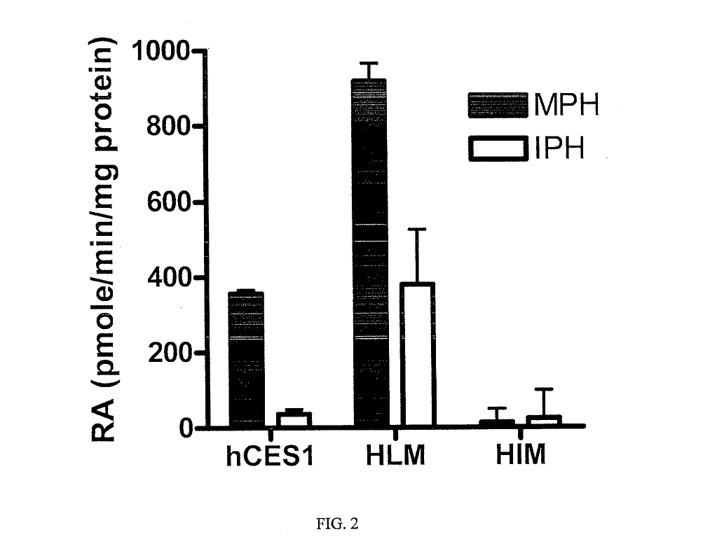

Hydrolysis of IPH and MPH. dl-MPH has consistently been found to be resistant to hydrolysis via hCES2. Although hCES1 is abundantly expressed in the human liver and is purported to account for the majority of hydrolytic activity within the organ, hCES2 is the predominant hydrolase in the human intestine. In the present study, hydrolytic properties of MPH and IPH were investigated in parallel by incubation of each substrate with recombinant hCES1, as well as HLM and HIM preparations. The results indicated that the catalytic efficiency of hCES 1-mediated hydrolysis upon MPH is approximately 10-fold higher than for IPH (FIG. 2). IPH is the most resistant substrate to HLM (FIG. 2). In contrast, catalytic activity of HIM is extremely low with regard to both MPH and IPH hydrolysis (FIG. 2).

In summary, the present data demonstrate that IPH is a poor substrate of hCES1, which is in stark contrast to MPH. Nevertheless, albeit at a markedly lower rate, IPH was predominantly metabolized (i.e., hydrolyzed) by hCES 1 with little to no contribution from hCES2, an observation similar to that which that is known to be the case with MPH and established structure-activity relations for hCES 1 and its ester substrates (Ross and Crow, 2007).

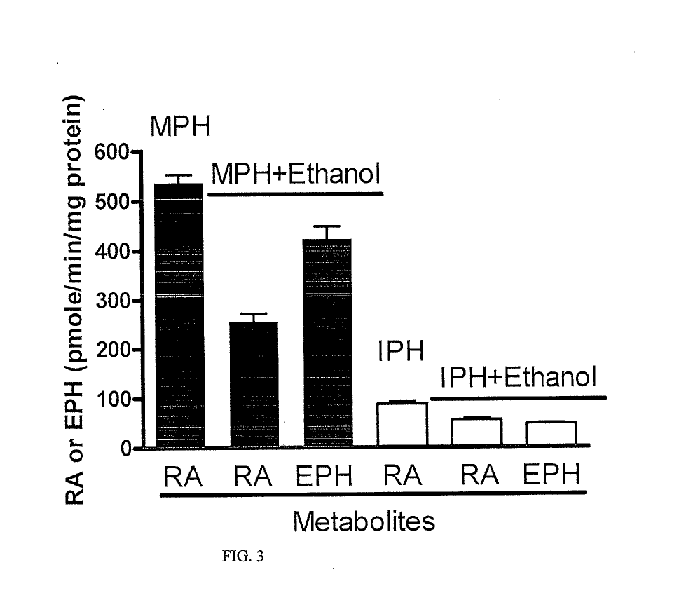

Transesterification Potential of Isopropylphenidate versus Methylphenidate. Consistent with the inventors' previous observations, the efficiency of dl-MPH hydrolysis mediated by hCES 1 was significantly higher than that of IPH. Although ethanol was included in the reaction system, MPH was still efficiently converted to EPH via hCES 1 with a velocity of 423.3±44.4 pmole/min/mg protein under the described experimental conditions. In contrast, compared to MPH, IPH displayed great resistance to hCES1-mediated transesterification, exhibiting a velocity of 47.5±3.3 pmole/min/mg protein (FIG. 3). Thus, IPH is a poor substrate of both hydrolysis and transesterification reactions catalyzed by hCES 1. Furthermore, no EPH formation was observed following the incubation of MPH and IPH with ethanol when hCES1 was not present. In addition to yielding the novel transesterification metabolite EPH when combined with MPH, ethanol also significantly decreased the formation of the hydrolytic product RA following incubation with MPH or IPH.

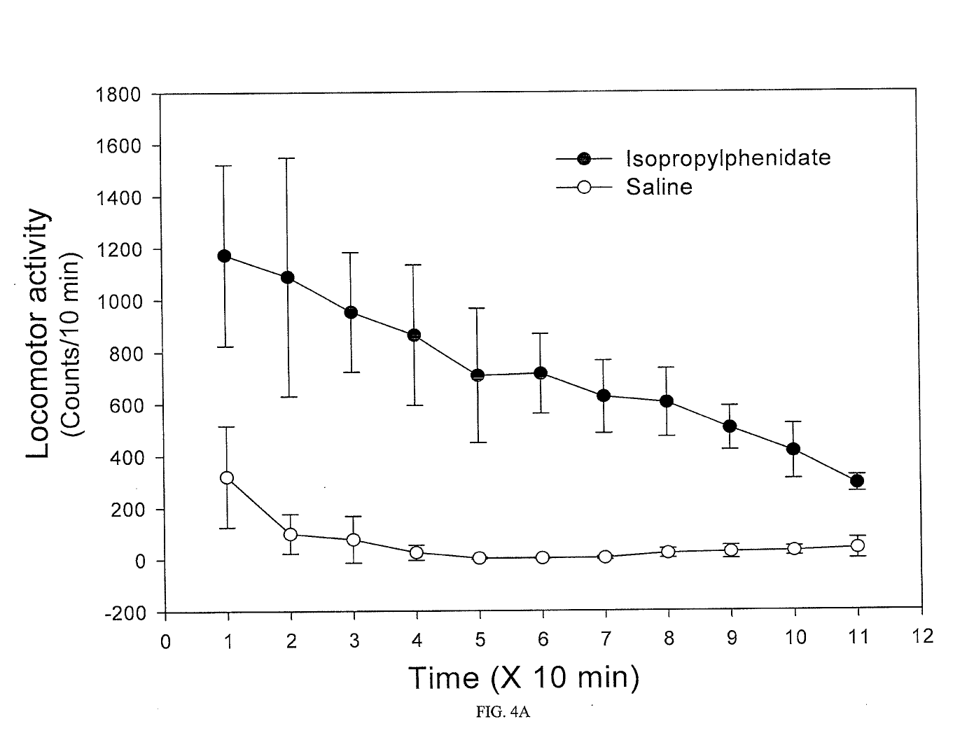

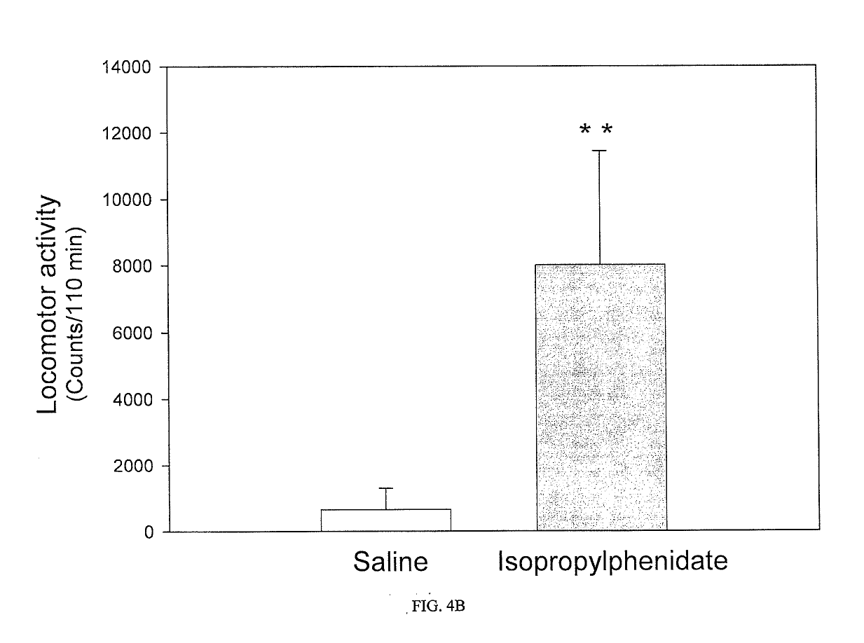

Rat Locomotor Activity. The time course of locomotor response over a 120 min period following i.p. administration of IPH (10 mg/kg) versus saline is shown in FIG. 4A. As expected, IPH produced robust effects on locomotor activity in rats compared to saline injections at every 10 min period recorded post-dosing with a mean of nearly 1200 counts recorded during the initial 10 min measurement. Also shown in FIG. 4B are the cumulative locomotor activity counts over the entire 120 min study period. In summary, racemic IPH administration significantly elevated the cumulative locomotor activity counts in comparison to saline-injected rats (p<0.01).

TABLE 1
Transporter Binding Affinity Study of MPH and IPH for the
Major Monoamine Transporters

| | | | Compound | |
|---|---|---|---|---|
| | | | Assessed | |
| | | | (10 μM) | |
| | | | (% inhibition | |
| | | | of control | |
| | | | specific | |
| Monoamine | | Reference | binding) | |
| Transporter | Ligand | Compound | IPH | MPH |
| DAT (h) | [³H]GBR-12935 | BTCP | 97 | 101 |
| NET (h) | [³H]nisoxetine | protriptyline | 27 | 88 |
| SERT (h) | [³H]imipramine | imipramine | 20 | 6 |

DAT = dopamine transporter
NET = norepinephrine transporter
SERT = serotonin transporter
BTCP = N-[1-(Benzo[b]thien-2-yl-cyclohexyl)]piperidine;
[³H]GBR-12935 = 1-[2-(Diphenylmethoxy)ethyl]-4-(3-phenylpropyl)piperazine,
MPH = racemic (dl) methylphenidate,
EPH = racemic ethylphenidate,
IPH = racemic isopropylphenidate

TABLE 2
Cellular Uptake Studies of MPH and IPH

| | | Compound | |
|---|---|---|---|
| | | Assessed | |
| | | (10 μM) | |
| | | (% inhibition | |
| | | of control | |
| | | specific | |
| Cellular Uptake | Measured | binding) | |
| Assay/Substrate | Parameter | IPH | MPH |
| Dopamine/[³H]DA | [³H]DA incorporation | 96 | 90 |
| | into synaptosomes | | |
| Norepinephrine/[³H]NE | [³H]NE incorporation | 62 | 117 |
| | into synaptosomes | | |
| Serotonin/[³H]5-HT | [³H]5-HT incorporation | 28 | 35 |
| | into synaptosomes | | |

### Example 3

### Discussion

Evaluation of the binding affinities of both compounds for DAT, NET, and SERT revealed a number of differences between these congeners. Overall, the transporter binding data generated for the prototype “phenidate” compound, MPH, was consistent with the majority of published in vitro reports conducting similar assessments (Markowitz and Patrick, 2008). Both tested compounds showed similar and significant binding for DAT, and little interaction with SERT (Table 1). With regard to NET, it was noted that IPH exhibited substantially less binding affinity than MPH. The results of the complementary cellular functional studies indicated that both tested agents produce a high degree of DA uptake inhibition and little difference in this action is noted between the two (Table 2). Uptake of NE was noted to be significantly lower for IPH relative to MPH. Lastly, 5-HT uptake was not significantly influenced by either of the agents.

Thus, in totality, basic pharmacological screening of monoamine transporters and cellular uptake studies suggest that IPH is a primarily dopaminergic compound with significantly less noradrenergic activity than MPH. Although there is substantial evidence of a noradrenergic component to both ADHD pathophysiology as well as its pharmacotherapy, the prevailing view is that worrisome cardiovascular side effects (increased heart rate and blood pressures) associated with MPH as well as amphetamines are primarily mediated by the noradrenergic component of these psychostimulant's actions through NE stimulation. Accordingly, an agent which appears to have less noradrenergic activity, such as IPH, could potentially provide an improved safety profile relative to existing drugs (e.g., MPH) presently employed in ADHD treatment.

The results of enzymatic hydrolysis experiments, which were conducted using transfected cells over-expressing hCES1, HLM, and HIM, also produced substantially different results for the IPH compound relative to MPH. The data generated in the present study demonstrate that IPH is a relatively poor substrate of hCES1, which was in stark contrast to that observed for MPH (FIG. 3). Nevertheless, albeit at a markedly lower rate, IPH was predominantly metabolized (i.e., hydrolyzed) by hCES1 to the RA, a metabolite common to the two compounds, with little to no contribution from hCES2. The reason for the less efficient hydrolysis of IPH relative to MPH cannot be stated with certainty. However it is hypothesized the bulkier isopropyl substitution provides sufficient steric hindrance to the active site of hCES1 relative to MPH. This is consistent with current theory on structural requirements for hCES1 substrates dictate that those molecules esterified by a small alcohol group and also contain a large acyl group (e.g., MPH). Conversely, hCES2 tends to show greater catalytic activity toward structures with larger alcohol groups and smaller acyl groups (Imai et al. 2006).

In the present investigation, i.p. administration of racemic IPH to rats produced potent locomotor activity effects, as consistent with the pharmacology of MPH and other DAT-active psychostimulants that have proven useful in the management of ADHD. These data add to existing in vitro data suggesting IPH to be a significantly active CNS compound at mg/kg doses similar to those used in assessments of MPH on producing classic stimulant-induced behavioral responses in the rat (Patrick et al., 1987). Furthermore, when compared to the earlier study by Patrick and associates (1987) characterizing the pharmacology of MPH isomers, and under identical experimental conditions, racemic IPH dosed at 10 mg/kg appeared to produce more potent and sustained locomotor responses than that of d-MPH dosed at 5 mg/kg. Additionally, an earlier study in which MPH was introduced intracerebroventricularly into Sprague-Dawley rats, locomotor activity as measured by the same method employed in the present study lasted only 60 min (Patrick et al., 1981). Although an acknowledged limitation of the present study was the lack of an active (i.e., MPH-treated) comparator group of animals, study conditions were identical to those previously employed (Patrick et al., 1987).

The present report provides in vitro evidence that IPH has a high affinity for DAT as well as potent effects on cellular uptake of DA but unlike MPH has only minor effects on NE which may provide for a more desirable safety/toxicity profile since noradrenergic effects are generally viewed as producing autonomic responses which may be undesirable (e.g. hypertension). Additionally, a substantially slower rate of enzymatic hydrolysis via the primary metabolic pathway mediated by hCES1 is noted relative to MPH. This suggests a longer duration of action and/or wider dosing intervals could be utilized which is viewed as necessary in the current treatment of ADHD with MPH. Finally, in vivo studies clearly demonstrate potent stimulating effects on locomotor activity in IPH dosed rats similar to that typically observed following MPH or amphetamine dosing utilizing a standard and widely accepted behavioral testing paradigm indicative of psychostimulant activity. Similar locomotor activity responses have been consistently reported in the published biomedical literature with the most commonly employed agents in the pharmacological treatment of ADHD. Taken together, these data are consistent with IPH providing a prolonged duration of action, reduced drug interaction liability, production of inactive/non-toxic metabolites, and a more selective dopaminergic effect to improve the pharmacotherapy of ADHD and other CNS disorders whose underlying etiologies are prominently based on dopaminergic dysfunction, or otherwise known to be improved by pharmacotherapy with dopaminergic medications.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference:
U.S. Pat. No. 6,242,464
Biederman, Biol. Psychiatry, 57:1215-1220, 2005.
Faraone et al., World Psychiatry, 2:104-113, 2003.
Greenhill et al., J. Am. Acad. Child. Adolesc. Psychiatry, 41(2):26S-49S, 2002.
Imai et al., Drug Metab. Dispos., 34:1734-1741, 2006.
Lazarou et al., JAMA, 279:1200-1205, 1998.
Markowitz et al., J. Clin. Psychopharmacol., 28:S54-S61, 2008.
Markowitz et al., Pharmacotherapy, 23:1281-1299, 2003.
Markowitz et al., J. Clin. Psychopharmacol., 19:362-6, 1999.
Markowitz et al., Drug Metab. Dispos., 28:620-24, 2000.
McConnell et al., Internat. J. Pharmaceut., 364:213-26, 2008.
Patrick et al., Hum. Psychopharmacol., 12:527-46, 1997.
Patrick et al., Clin. Pharmacol. Ther., 81(3):346-53, 2007.
Patrick et al., Human Psychopharm. Clin. Exper., 24:1-17, 2009.
Patrick et al., J. Med. Chem., 29:1237-40, 1981.
Patrick et al., J. Med. Chem., 48:2876-2881, 2005.
Patrick et al., J. Pharmacol. Exp. Ther., 241:152-158, 1987.
Portoghese and Malspeis, J. Pharmacol. Sci., 50:494-501, 1961.
Ross and Crow, J. Biochem. Mol. Toxicol., 21(4):187-196, 2007.
Schweri et al., J. Neurochem., 45:1062-1070, 1985.
Singh, Chem. Rev., 100:925-1024, 2000.
Thai et al., Drug Metab. Dispos., 27:645-650, 1999.
Williard et al., Behav. Pharmacol., 18:39-51, 2007.
Zhu et al., Am. J. Hum. Genetics, 82:1241-8, 2008.

1. A method of treating a subject with attention-deficit/hyperactivity disorder (ADHD) comprising administering to said subject a pharmaceutically acceptable form of d- and/or dl-threo-isopropylphenidate or a salt thereof.

2. The method of claim 1, wherein said subject is a human.

3. The method of claim 1, wherein said subject is a child or adolescent.

4. The method of claim 1, wherein said subject is an adult.

5. The method of claim 1, wherein said subject further suffers from a comorbid disorder.

6. The method of claim 1, wherein d- and/or dl-threo-isopropylphenidate or salt thereof is administered to said subject in a unit dosage form of 1 to 200 milligrams.

7. The method of claim 6, wherein said unit dosage form is about 10 to 100 milligrams.

8. The method of claim 6, wherein said subject is provided said unit dosage form every 4-24 hours.

9. The method of claim 1, wherein said d- and/or dl-threo-isopropylphenidate or salt thereof is administered orally.

10. The method of claim 1, where said d- and/or dl-threo-isopropylphenidate or salt thereof is administered as an injectable solution, a transdermal patch, a capsule, a tablet, an intranasal or sublingual spray, a syrup, or a solution.

11. The method of claim 1, wherein said subject consumes alcohol during treatment with d- and/or dl-threo-isopropylphenidate or salt thereof.

12. The method of claim 1, wherein said cells of said subject comprise a mutated human carboxylesterase-1 gene.

13. The method of claim 1, wherein d- and/or dl-threo-isopropylphenidate or salt thereof is substantially d-isopropylphenidate.

14. The method of claim 1, wherein d- and/or dl-threo-isopropylphenidate or salt thereof is substantially dl-isopropylphenidate.

15. The method of claim 1, wherein d- and/or dl-threo-isopropylphenidate or salt thereof is a racemic mixture.

16. The method of claim 1, wherein d- and/or dl-threo-isopropylphenidate salt is a hydrochloride, hydrobromide, sulfate, aspartate, saccharate, succinate, tartrate, mesylate, or palmitate salt.

17. The method of claim 1, wherein said subject is treated with a second ADHD therapy.

18. The method of claim 17, wherein said second ADHD therapy is methylphenidate.

19. A pharmaceutical formulation comprising (a) d- and/or dl-threo-isopropylphenidate or salt thereof and (b) an opiate and/or a sedating anti-psychotic.

20. (canceled)

21. A pharmaceutical formulation comprising (a) d- and/or dl-threo-isopropylphenidate or salt thereof and (b) methylphenidate.

22. (canceled)

23. A method of treating fatigue or somnolence in a subject comprising administering to said subject a pharmaceutically acceptable form of d- and/or dl-threo-isopropylphenidate or salt thereof.

24-42. (canceled)
